(19) United States
(12) Patent Application Publication
(10) Pub. No.: US 2022/0000889 A1
(43) Pub. Date: Jan. 6, 2022

(54) INOSITOL PHOSPHATE COMPOUNDS FOR USE IN INCREASING TISSULAR PERFUSION

(71) Applicant: SANIFIT THERAPEUTICS, S.A., Palma de Mallorca (ES)

(72) Inventors: Mohamad Firas BASSISSI, Palma de Mallorca (ES); Carolina SALCEDO ROCA, Palma de Mallorca (ES); Joan PERELLÓ BESTARD, Palma de Mallorca (ES); Miquel David FERRER REYNÉS, Palma de Mallorca (ES); María Del Mar PÉREZ FERRER, Palma de Mallorca (ES)

(73) Assignee: SANIFIT THERAPEUTICS, S.A., Palma de Mallorca (ES)

(21) Appl. No.: 17/381,052

(22) Filed: Jul. 20, 2021

Related U.S. Application Data
(63) Continuation of application No. PCT/ES2020/070070, filed on Jan. 30, 2020.
(60) Provisional application No. 62/913,259, filed on Oct. 10, 2019.

(30) Foreign Application Priority Data
Jan. 30, 2019 (EP) 19382061.0

(51) Int. Cl. A61K 31/6615; A61P 9/10

(52) U.S. Cl. CPC A61K 31/6615; A61P 9/10

(57) ABSTRACT

The present invention relates to inositol phosphates, analogs, derivatives and pharmaceutically acceptable salts thereof, for use in increasing tissular perfusion and/or oxygenation in a subject in need thereof, in particular peripheral arterial disease. The present invention also relates to pharmaceutical compositions comprising said inositol phosphates, analogs, derivatives and pharmaceutically acceptable salts thereof, and their use in increasing tissular perfusion and/or oxygenation and for treating and preventing peripheral arterial disease.

### RELATED APPLICATIONS

This application is a continuation of International Application No. PCT/ES2020/070070, filed on Jan. 30, 2020, which claims the priority benefits of European Application No. EP19382061.0, filed on Jan. 30, 2019, and U.S. Provisional Application No. 62/913,259, filed on Oct. 10, 2019, all of which are herein incorporated by reference in their entireties.

### FIELD OF THE INVENTION

The present invention relates to the use of inositol phosphates (IP), their analogs and derivatives for increasing tissular perfusion and/or oxygenation. The present invention also relates to pharmaceutical compositions comprising said IP, their analogs and derivatives and their use in animal and human health.

### BACKGROUND ART

Peripheral arterial disease (PAD) is a common disorder characterized by stenosis and/or obstruction of the lower limb arteries leading to a decreased muscle perfusion and oxygenation. PAD represents a major public health issue and poses a high risk of long-term suffering. PAD increases the risk of tissue death (gangrene), amputation and premature death.

PAD is the result of ischemia in the lower limbs. Its principal cause is atherosclerosis. In its mild form, PAD may be limited to intermittent claudication and pain in the lower extremities. Lower extremity PAD is a major cause of disability and mobility loss in older men and women and has a decisive impact on quality of life.

The prevalence of PAD is ˜12% in the adult population. This prevalence increases to over 20% in the population group with more than 70 years of age. PAD now affects more than eight million men and women in the United States only. It is estimated that more than 200 million individuals suffer PAD worldwide. The prevalence of PAD is likely to increase in the near future as the general population grows older and the incidence of obesity-related type 2 diabetes increases. Cigarette smoking is also another significant risk factor. PAD patients have increased rates of cardiovascular (CV) morbidity and mortality, faster rates of functional decline, and increased rates of mobility loss compared to the general population.

The protocol objectives for the treatment of PAD patients include reducing CV event rates, improving functional performance, and preventing functional decline and the loss of mobility. Restoring or improving blood perfusion to the limbs can help to achieve these goals.

While endovascular and lower extremity revascularization procedures significantly improve walking performance in PAD patients, revascularization procedures are not therapeutic options for many of them, either due to the presence of comorbid diseases or because the location and type of atherosclerotic disease in the lower extremities is not amenable to revascularization. Revascularization is invasive, costly, and associated with risks, especially for older patients. For these reasons, there is a demand from clinicians for medical therapies that improve lower extremity functioning in PAD patients which are effective, accessible and well tolerated.

At the present, only two medicaments have been approved by the Federal Drug Administration (FDA) for improving walking performance in people with PAD: pentoxifylline (1984) and cilostazol (1999). No new drugs have been approved for treating intermittent claudication since then. Furthermore, in recent studies in PAD patients, pentoxifylline has not significantly improved intermittent claudication symptoms or maximal walking distance more than placebo. Recently published clinical practice guidelines recommend against prescription of pentoxifylline for intermittent claudication symptoms, due to a lack of therapeutic benefit.

Cilostazol is a phosphodiesterase inhibitor that provides approximately 25% to 40% improvement in treadmill walking performance in people with symptomatic PAD. Cilostazol is a phosphodiesterase type 3 inhibitor that acts by increasing the intracellular concentration of cyclic adenosine monophosphate; in the process, the drug suppresses platelet aggregation and serves as a direct arterial vasodilator, improving of blood perfusion. However, the mechanism by which cilostazol improves walking ability in PAD patients remains unclear.

Side effects of cilostazol include headache, diarrhea, palpitations, and lightheadedness. There is a black box warning against prescribing cilostazol to subjects with history of cardiovascular diseases. Cilostazol should not be administered to PAD patients who also have heart failure. Cilostazol interacts with drugs prescribed regularly to patients with renal impairment or cardiovascular diseases, such as cinacalcet, clopidogrel and ibandronate, thus increasing the risk of an adverse reaction to these patients arising from the combined use cilostazol with other drugs.

In conclusion, medical therapies for symptomatic relief are limited, surgical or endovascular interventions are useful for some individuals, but long-term results are often disappointing. As a result, there is a need for developing more effective and safer new therapies for treating PAD.

### SUMMARY OF THE INVENTION

In a first aspect, the present invention relates to a compound of general formula I, or a pharmaceutically acceptable salt thereof, for use in increasing tissular perfusion and/or oxygenation in a subject in need thereof.

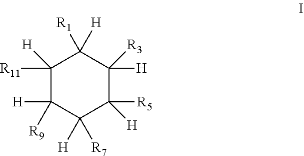

where R₁, R₃, R₅, R₇, R₉ and R₁₁ are independently selected from OH, a radical of formula II, III, IV and a heterologous moiety:

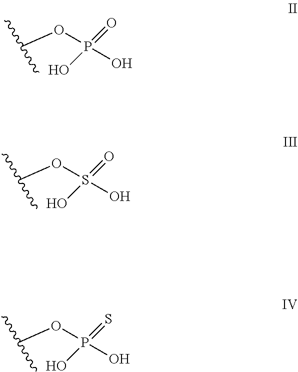

with the condition that:
at least one of R₁, R₃, R₅, R₇, R₉ and R₁₁ is selected from a radical of formula II, III and IV, and
zero, one, two or three of R₁, R₃, R₅, R₇, R₉ and R₁₁ is a heterologous moiety.

In another aspect, the present invention relates to a compound of general formula I, as defined above, for use in the treatment or prevention of ischemia in a subject in need thereof. In a version of this aspect, the invention refers to a compound of general formula I, as described above, for use in the treatment or prevention of an ischemia-related disease or condition in a subject in need thereof.

In some aspects, the present invention refers to a compound of general formula I, as defined above, wherein the heterologous moiety is selected from a radical of formula V, a radical of formula VI and a radical of formula VII:

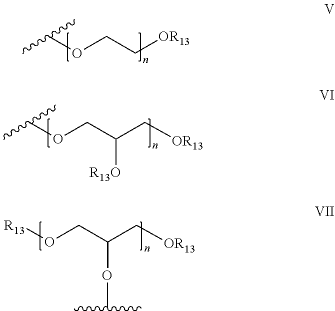

and wherein n is an integer in the range from 2 to 200, and R₁₃ is selected from H, methyl or ethyl.

In a further aspect, the invention also relates to a method for increasing tissular perfusion and/or oxygenation which comprises administering a therapeutically effective amount of a compound of formula I, as defined above, together with pharmaceutically acceptable excipients or carriers, to a subject in need thereof. This aspect may also be formulated as the use of a compound of formula I, as defined above, for the manufacture of a medicament for increasing tissular perfusion and/or oxygenation in a subject in need thereof.

In another aspect, the invention also relates to a method for treating or preventing ischemia and/or an ischemia-related disease or condition which comprises administering a therapeutically effective amount of a compound of formula I, as defined above, together with pharmaceutically acceptable excipients or carriers, to a subject in need thereof. This aspect may also be formulated as the use of a compound of formula I, as defined above, for the manufacture of a medicament for treating or preventing ischemia and/or an ischemia-related disease or condition in a subject in need thereof.

In a further aspect, the invention relates to a method for treating or preventing peripheral arterial disease which comprises administering a therapeutically effective amount of a compound of formula I, as defined above, together with pharmaceutically acceptable excipients or carriers, to a subject in need thereof. This aspect may also be formulated as the use of a compound of formula I, as defined above, for the manufacture of a medicament for treating or preventing peripheral arterial disease in a subject in need thereof.

The compounds of the present invention are particularly useful for increasing tissular perfusion and/or oxygenation in the lower limbs and, especially, for the treatment or prevention of peripheral artery disease (PAD) and closely related conditions such as critical limb ischemia (CLI). These compounds also exhibit many advantageous properties (e.g., better safety profile) in comparison to cilostazol, the reference drug currently indicated for the treatment of PAD.

The invention also provides a pharmaceutical composition comprising at least one compound of formula I, as defined above, for use in: (i) increasing tissular perfusion and/or oxygenation, (ii) treating or preventing ischemia and/or an ischemia-related disease, and/or (iii) treating or preventing PAD in a subject in need thereof. This aspect may also be formulated as the use of a pharmaceutical composition comprising at least one compound of formula I, as defined above, for the manufacture of a medicament for: (i) increasing tissular perfusion and/or oxygenation, (ii) treating or preventing ischemia and/or an ischemia-related disease, and/or (iii) treating or preventing PAD in a subject in need thereof.

### BRIEF DESCRIPTION OF THE DRAWINGS

FIG. 1 shows representative examples of inositol phosphate analogs in which two out of six X are OPSO₂²⁻ and the remaining X are OSO₃. Two specific forms of 4,6-di-(O-thiophosphate)-inositol-1,2,3,5-tetra-O-sulfate are shown.

FIG. 2 shows inositol phosphate analogs and inositol phosphate derivatives that can be used to practice the methods of the present invention. The molecules shown are myo-inositol-pentakisphosphate-2-PEG400, myo-inositol hexakissulfate (myo-inositol hexasulfate), and scyllo-myo-inositol hexakissulfate (scyllo-inositol hexasulfate)

FIG. 3 shows inositol phosphate analogs and inositol phosphate derivatives that can be used to practice the methods of the present invention. X represent independently phosphorus and/or sulfur containing groups (e.g., phosphate, sulfate, or thiophosphate). R¹ represents a heterologous moiety (e.g., PEG or PG).

FIG. 4 shows exemplary inositol phosphate analogs and inositol phosphate derivatives that can be used to practice the methods of the present invention. R¹ represents a heterologous moiety (e.g., PEG or PG). n can be between 2 and 200.

FIG. 5 shows exemplary inositol phosphate analogs and inositol phosphate derivatives that can be used to practice the methods of the present invention. n can be between 2 and 200.

FIG. 6 shows exemplary inositol phosphate analogs and inositol phosphate derivatives that can be used to practice the methods of the present invention. n can be between 2 and 200.

FIG. 7 shows blood flow in posterior limbs in a rat model at D0 measured by doppler laser imaging. Blood flow is shown in normalized perfusion units (PU). Normalization is obtained by comparing the raw data to the group 1 data at D0.

FIG. 8 shows blood flow in posterior limbs in a rat model at D6 measured by doppler laser imaging. Blood flow is shown in normalized perfusion units (PU). Normalization is obtained by comparing the raw data to the group 1 data at D6.

FIG. 9 shows blood flow in posterior limbs in a rat model at D12 measured by doppler laser imaging. Blood flow is shown in normalized perfusion units (PU). Normalization is obtained by comparing the raw data to the group 1 data at D12.

FIG. 10 shows inhibition percentage of aorta calcification in a VitD rat model at D12. Calcium level at sacrifice time was measured by ICP-OES.

FIG. 11 shows blood flow in posterior limbs in a rat model at D12 and D18 (6 days after the interruption of treatment) measured by doppler laser imaging. Blood flow is shown in normalized perfusion units (PU). Normalization is obtained by comparing the raw data to the group 1 data at D12 and D18.

FIG. 12 shows (A) Maximum Walking Distance (MWD) and (B) Maximum Walking Time (MWT) in a rat model at D10 measured by treadmill test. Maximum Walking Distance is shown in meters (m) and Maximum Walking Time in minutes (min).

FIG. 13 shows Maximum Walking Distance (MWD) in a rat model at D17 (5 days after the interruption of treatment) measured by treadmill test. Maximal Walking Distance is shown in meters (m) up to 40 min of walking time.

FIG. 14 shows inhibition percentage of aorta calcification in a VitD rat model at D24 (12 days after the interruption of treatment). Calcium level at sacrifice time was measured by ICP-OES.

FIG. 15 shows blood flow in posterior limbs in a rat model at D0, D5, and D13 (8 days after starting treatment) measured by doppler laser imaging. Blood flow is shown in normalized perfusion units (PU). Normalization is obtained by comparing the raw data to the group 1 data at D0, D5, and D13.

FIG. 16 shows (A) Maximum Walking Distance (MWD) and (B) Maximum Walking Time (MWT) in a rat model at D11 (7 days after starting treatment) measured by treadmill test. Maximum Walking Distance is shown in meters (m) and Maximum Walking Time in minutes (min).

FIG. 17 shows inhibition percentage of femoral arteries calcification in a VitD rat model at D13 (9 days after starting treatment). Calcium level at sacrifice time was measured by ICP-OES.

### DETAILED DESCRIPTION OF THE INVENTION

The present invention provides compounds, pharmaceutical compositions, methods and routes of administration for use in increasing tissular perfusion and/or oxygenation. The invention also provides compounds, pharmaceutical compositions, methods and routes of administration for use in the treatment or prevention of ischemia and ischemia-related diseases and conditions.

The compounds of the present invention are particularly useful for increasing tissular perfusion and/or oxygenation in the lower limbs and, especially, for the treatment or prevention of peripheral artery disease (PAD) and related conditions such as critical limb ischemia (CLI). These compounds also exhibit many advantageous properties in comparison to other approved drugs for the treatment of PAD and CLI.

### 1. Definitions of General Terms and Expressions

The present invention includes embodiments in which exactly one member of the group is present in, employed in, or otherwise relevant to a given product or process. It also includes embodiments in which more than one, or all of the group members are present in, employed in, or otherwise relevant to a given product or process.

Unless defined otherwise, all technical and scientific terms used herein have the same meaning as commonly understood by a skilled person in the art to which this description is related. For example, the Pei-Show J, Concise Dictionary of Biomedicine and Molecular Biology, 2nd Ed. (CRC Press, Boca Raton, Fla., USA 2002); Lackie J, The Dictionary of Cell and Molecular Biology, 5^{th} Ed. (Academic Press, Cambridge, Mass., USA 2013) and Cammack R, et al., Oxford Dictionary of Biochemistry and Molecular Biology, 2^{nd} Ed., (Oxford University Press, Oxford, G B, 2006) provide the skilled person with a general dictionary of many of the terms used in this description.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention.

Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

The term “and/or” as used herein is to be taken as the specific invention of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “around” as used herein and as applied to one or more values of interest, refers to a value that is similar to a stated reference value. In certain aspects, the term “around” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The terms “critical limb ischemia” or “CLI” as used herein refer to a severe obstruction of the arteries which markedly reduces blood flow to the extremities and progresses to the point of severe pain and even skin ulcers, sores, or gangrene. Critical limb ischemia is a very severe condition of peripheral artery disease. In some aspects, the administration of the inositol phosphate of the present invention (e.g., myo-inositol hexaphosphate) to a subject in need thereof improves its capacity for walking faster and for longer distances in comparison when untreated.

The term “compound” as used herein is meant to include all isomers and isotopes of the structure depicted. As used herein, the term “isomer” means any geometric isomer, tautomer, zwitterion, stereoisomer, enantiomer, or diastereomer of a compound. Compounds can include one or more chiral centers and/or double bonds and can thus exist as stereoisomers, such as double-bond isomers (i.e., geometric E/Z isomers) or diastereomers (e.g., enantiomers (i.e., (+) or (−)) or cis/trans isomers). The present invention encompasses any and all isomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. A compound, salt, or complex of the present invention can be prepared in combination with solvent or water molecules to form solvates and hydrates by routine methods.

The term “cilostazol” as used herein, refers to 6-[4-(1-cyclohexyl-1H-tetrazol-5yl)butoxy]-3,4-dihydro-2(1H)-quinolinone [CAS-73963-72-1], a quinolinone derivative that inhibits cellular phosphodiesterase. The molecular formula and weight of cilostazol are C₂₀H₂₇N₅O₂ and 369.46 g/mol, respectively. Its structural formula is:

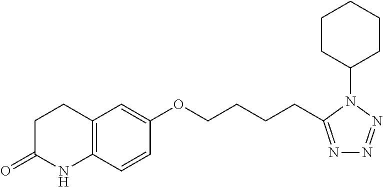 [SMILES: O=C1CCc2cc(OCCCCc3nnnn3C3CCCCC3)ccc2N1]

The term “effective amount” as used herein, and the related terms “effective dose” and “effective dosage” in reference to (i) a compound of a general formula I (e.g. an inositol phosphate, an inositol phosphate analog, an inositol phosphate derivative, or a combination thereof), or (ii) a pharmaceutical composition comprising at least one of the item (i) compounds, is that amount sufficient to effect beneficial or desired results. In some embodiments, the beneficial or desired results are, for example, clinical results, and, as such, an “effective amount” depends upon the context in which it is being applied. In the context of administering a therapeutic agent that increases tissular perfusion and/or oxygenation, an effective amount of a therapeutic agent is, for example, an amount sufficient for (a) augmenting tissular perfusion in a specific area, (b) stopping, reducing, slowing the progression or reverting ischemia in a specific area or (c) improving the mobility or walking ability (e.g. velocity, distance) in a subject, as compared to the same parameters observed in the subject before the administration of the therapeutic agent, or in a population of control subjects without administration of the therapeutic agent.

The term “ischemia” as used herein refers to a restriction in blood supply to tissues, causing a shortage of oxygen that is required for maintaining cellular metabolism. Ischemia comprises not only insufficiency of oxygen, but also reduced availability of nutrients and inadequate removal of metabolic wastes. Ischemia can be partial (poor perfusion) or total.

The terms “maximal walking distance” or “MWD” as used herein refer to the distance at which a subject could not continue to walk unassisted due to exhaustion or extreme pain. In the context of assessing an increase in MWD, said increment is evaluated by comparing a subject's MWD values before and after treatment with a therapeutic agent, or by comparing the subject's MWD values after treatment with a population of control subjects untreated with the therapeutic agent.

The terms “maximal walking time” or “MWT” as used herein refer to the time at which a subject could not continue to walk unassisted due to exhaustion or extreme pain. In the context of assessing an increase in MWT, said increment is evaluated by comparing a subject's MWT values before and after treatment with a therapeutic agent, or by comparing the subject's MWT values after treatment with a population of control subjects untreated with the therapeutic agent.

The terms “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and, intrasternal injection and infusion (e.g., kidney dialytic infusion).

The terms “peripheral arterial disease” or “PAD” as used herein refer to a narrowing of the peripheral arteries to the legs (most commonly), stomach, arms, and head. Symptoms include intermittent claudication (e.g., leg pain when walking which resolves with rest), skin ulcers, bluish skin, cold skin, or poor nail and hair growth.

The terms “prevent”, “preventing” and “prevention” as used herein refer to inhibiting the inception or decreasing the occurrence of a disease or condition in a subject (e.g., avoiding the development of ischemic tissue in the limbs).

The term “SNF472” as used herein refers to an intravenous myo-inositol hexaphosphate hexasodium formulation. SNF472 is manufactured by dissolving myo-inositol hexaphosphate hexasodium in saline solution, followed by pH adjustment and aseptic filtration. SNF472 is prepared at three different strengths: (a) (i) 20 mg/mL and (ii) 90 mg/mL in 5 mL single-use vials, formulated in saline solution, pH 5.8 to 6.2 and (b) 30 mg/L in 10 mL single-use vials, formulated in saline solution, pH 5.6 to 6.4.

The terms “subject”, “individual”, “animal” or “mammal” as used herein is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain aspects, the subject is a human subject. In some aspects, the subject is a human patient with a reduced tissular perfusion and/or oxygenation in the lower limb muscles or at risk of developing said condition. In some further aspects, the subject is a human patient with ischemia and/or an ischemia-related disease or condition, or at risk of developing said ischemia, ischemia-related disease or condition.

The term “substantially” as used herein refers to the qualitative condition of exhibiting total or near-total extent or degree of a characteristic or property of interest. A person skilled in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “tissular perfusion” as used herein refers to the flow of blood or other perfusate through the vessels of a specific tissue or organ. “Increase tissular perfusion” or “increasing tissular perfusion” as used herein relate to an increment in the blood flow in a specific tissue area in a subject after administering an inositol phosphate of the present invention as compared to the same parameters observed in the subject before the administration of said therapeutic agent, or in a population of control subjects without administration of said therapeutic agent.

The terms “treat” or “treatment” as used herein refer to the administration of compound or pharmaceutical composition of the present invention for (i) slowing, (ii) inhibiting the progression, (iii) stopping, or (iv) reverting the progression of a disease or condition after its clinical signs have appeared. Control of the disease progression is understood to mean the beneficial or desired clinical results that include, but are not limited to, reduction of the symptoms, reduction of the duration of the disease, stabilization of pathological states (specifically to avoid additional deterioration), delaying the progression of the disease, improving the pathological state and remission (both partial and total). The control of progression of the disease also involves an extension of survival compared with the expected survival if treatment was not applied. Within the context of the present invention, the terms “treat” and “treatment” refer specifically to (a) increasing tissular perfusion and/or oxygenation or (b) stopping, reducing, slowing the progression or reverting the development of ischemic tissue, especially in the lower limbs, or (c) improving the mobility or walking ability (e.g., velocity, distance, endurance) in a subject administered with the compounds or the pharmaceutical compositions of the present invention.

The term “walking ability” as used herein refers to the capacity of a subject for mobilizing autonomously without assistance. The parameters MWD and MWT are indicative of a subject's walking ability.

### 2. Compounds

The compounds for use in the present invention are inositol phosphates, as defined in the first aspect of the invention, as well as analogs and derivatives thereof. The term “inositol phosphate” as used herein refers to a compound with an inositol ring and one, two, three, four, five, or six phosphate groups, or a combination thereof. Myo-inositol hexaphosphate (IP6) is an exemplary inositol phosphate of the present invention. In some aspects, the inositol phosphate is pure (e.g., over 99% of the inositol phosphate species are the same species, for example, IP6) or substantially pure (e.g., at least 90%, 91%, 92%, 93%, 94%, 95%, 96%, 97%, 98% or 99% of the inositol phosphate species are the same species, for example, IP6). In some aspects, the inositol phosphate is a mixture, e.g., comprising variable amounts of IP1, IP2, IP3, IP4, IP5, and IP6. In some aspects, the inositol phosphate is a racemic mixture.

The invention also contemplates inositol phosphate analogs. “Inositol phosphate analog” as used herein refers to a compound that has a ring with different number of carbons with respect to an inositol ring (i.e., 5 or 7 carbons), and/or has at least one sulfate or thiophosphate group. For example, a compound comprising a ring with 5, 6, or 7 carbons and at least one phosphate, sulfate, or thiophosphate group would be considered an inositol phosphate analog.

The term “inositol phosphate derivative” as used herein refers to an inositol phosphate or inositol phosphate analog which contains a heterologous moiety (i.e., a group that is not a phosphate, a sulfate, or a thiophosphate). For example, an inositol pentasulfate comprising a polyethylene glycol heterologous moiety, or a myo-inositol hexaphosphate comprising a polyglycerol heterologous moiety would be considered inositol phosphate derivatives.

The term “heterologous moiety” as used herein refers to a radical in the compound of formula I which is not a phosphate, a sulfate, or a thiophosphate, and confers a desirable property to such compound. For example, a heterologous moiety (e.g., a polyglycerol or a polyethyleneglycol) can increase the solubility of the compound. In some aspects, a heterologous moiety can confer multiple desirable properties (e.g., polyglycerol and polyethyleneglycol can both increase the solubility of a compound and reduce the clearance rate of the compound).

The terms “inositol phosphate of the invention” and “inositol phosphate of the present invention” as used herein is a generic term encompassing “inositol phosphate”, “inositol phosphate analog”, “inositol phosphate derivative” and combinations thereof. In some aspects, the term “inositol phosphate of the present invention” encompasses compositions comprising an “inositol phosphate” an “inositol phosphate analog” an “inositol phosphate derivative” or a combination thereof, and at least one additional therapeutic agent. In some aspects, the additional therapeutic agent comprises cilostazol, pentoxifylline or a combination thereof.

Compounds of the present invention comprising a ring with 5, 6, or 7 carbons and at least one sulfate, or thiophosphate group but without a phosphate group would still be considered an “inositol phosphate analog” or an “inositol phosphate analog” in the context of the present invention. Thus, the term “inositol phosphate of the present invention” encompasses not only phosphate-containing compounds but also compounds without phosphate groups that comprise a ring with 5, 6, or 7 carbons and at least one sulfate, or thiophosphate group.

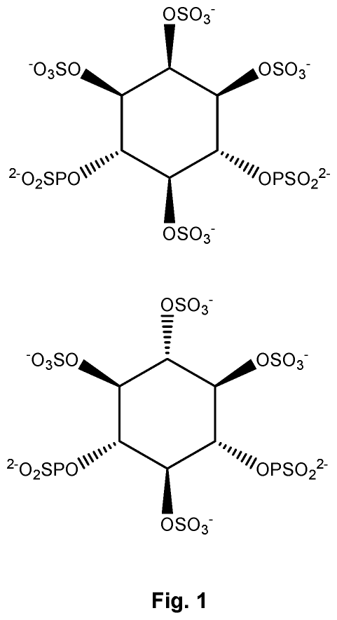

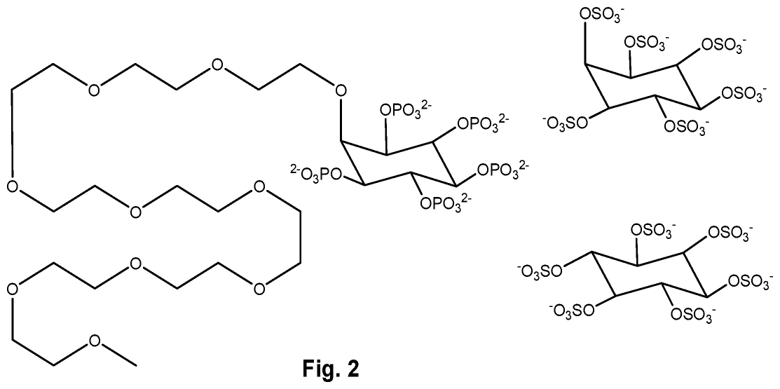

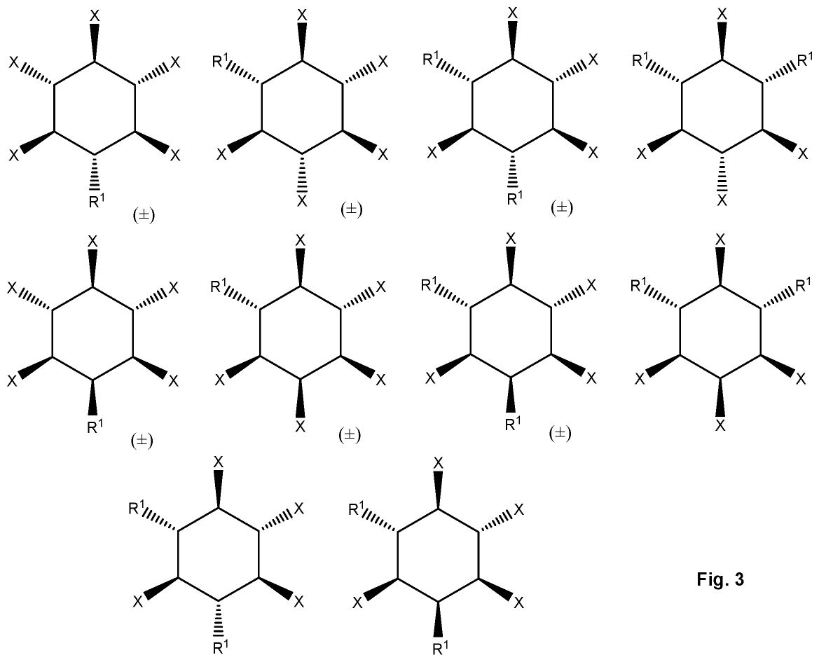

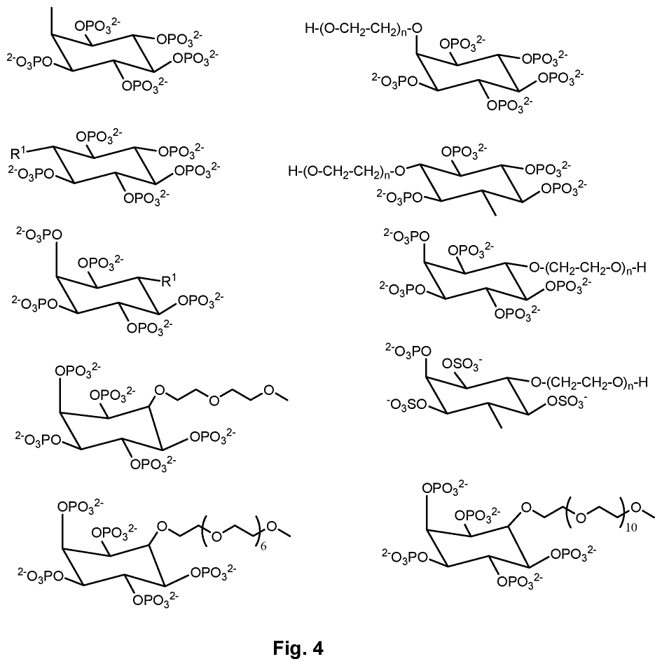

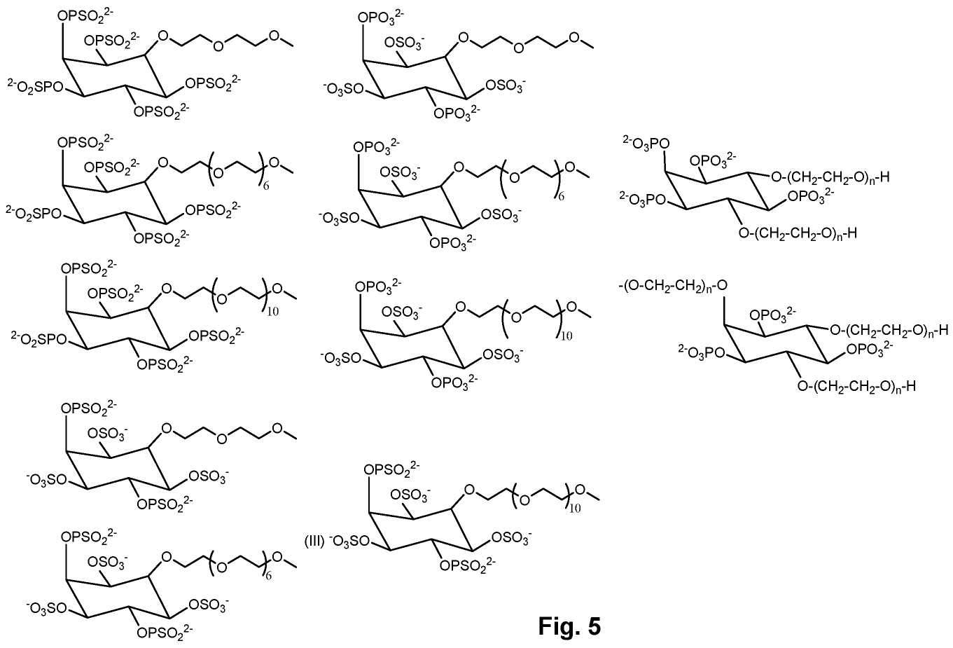

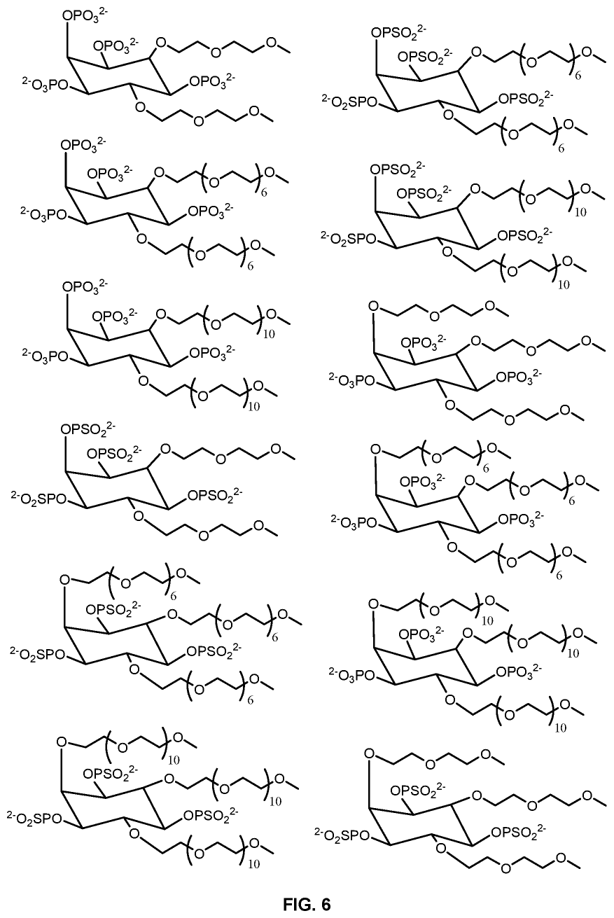

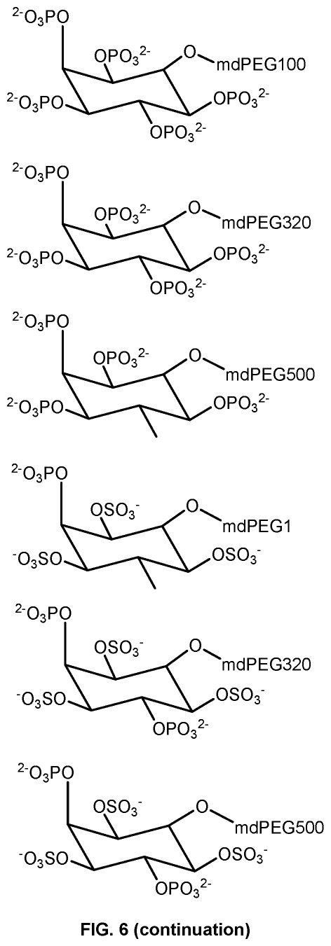

Representative inositol phosphates of the present invention are presented in FIGS. 1-6. FIG. 3 present numerous examples of inositol phosphates, all of them in the myo conformation. Besides myo-inositol, the other naturally occurring stereoisomers of inositol are scyllo-, muco-, 1D-chiro-, 1L-chiro-, neo-inositol, allo-, epi-, and cis-inositol. As their names denote, 1L- and 1D-chiro inositol are the only pair of inositol enantiomers, but they are enantiomers of each other, not of myo-inositol. It is to be understood that any exemplary inositol phosphate presented in the disclosure is not limited to the representative conformation displayed. Thus, for example, the examples presented in FIG. 3 would also encompass the corresponding equivalents in scyllo-, muco-, 1D-chiro-, 1L-chiro-, neo-inositol, allo-, epi-, and cis-inositol conformations. In its most stable conformation, the myo-inositol isomer assumes the chair conformation, which moves the maximum number of hydroxyls to the equatorial position, where they are farthest apart from each other. In this conformation, the natural myo isomer has a structure in which five of the six hydroxyls (the first, third, fourth, fifth, and sixth) are equatorial, whereas the second hydroxyl group is axial.

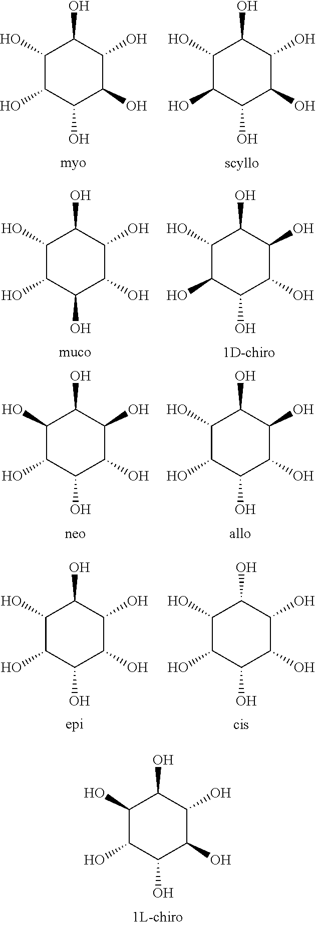

### 2.1. Inositol Phosphates, Analogs and Derivatives

In some aspects, at least one of R₁, R₃, R₅, R₇, R₉ and R₁₁ of the compound of general formula I independently represents H, —X, —OX, —NHX, —NX₂, —SX, —OSO₃HX, —OSO₃X₂ or a compound of formula II, formula III or formula IV, where each X independently represents H, C₁₋₃₀ alkyl, C₂₋₃₀ alkynyl or Cy₁, where C₁₋₃₀ alkyl, C₂₋₃₀ alkenyl and C₂₋₃₀ alkynyl are independently optionally substituted with one or more R₁₄ and where Cy₁ is optionally substituted by one or more R₁₅; Cy₁ represents a carbocyclic or heterocyclic three- to 10-membered ring, which can be saturated, partially unsaturated or aromatic, where said heterocycle has between one and four heteroatoms selected from amongst O, S and N, where said ring can be bound to the rest of the molecule via any available C atom and where Cy₁ is optionally fused to between one and four five- or six-membered rings, each saturated, partially unsaturated or aromatic, carbocyclic or heterocyclic, and where said fused heterocycle can contain one or two heteroatoms selected from amongst O, N and S; each R₁₃ independently represents H, C₁₋₃₀ alkyl, —NH₂, —NHC₁₋₃₀ alkyl or N(C₁₋₃₀ alkyl)₂, where each C₁₋₃₀ alkyl is independently optionally substituted with one or more halogen, —OH, —CN and —NO₂ groups; and each R₁₄ and R₁₅ independently represents OH, C₁₋₃₀ alkoxy, C₁₋₃₀ alkyithionyl, C₁₋₃₀ acyloxy, phosphate, halogen, trihalo C₁₋₃₀ alkyl, nitrile azide.

In some ulterior aspects, each X independently represents H, C₁₋₃₀ alkyl or Cy₁, where C₁₋₃₀ alkyl is optionally substituted by one or more R₁₄ and where Cy₁ is optionally substituted by one or more R₁₅; and each R₁₄ and R₁₅ independently represents —OH, C₁₋₃₀ alkoxy, C₁₋₃₀ alkyithionyl, C₁₋₃₀ acyloxy, phosphate, halogen, trihaloC₁₋₃₀alkyl, nitrile or azide. In some aspects, each X represents H, C₁₋₃₀alkyl or Cy₁. In some aspects, each X represents H.

In some additional aspects, at least one of radicals R₁, R₃, R₅, R₇, R₉ and R₁₁ independently represents a compound of formula II, formula III or formula IV, each R₁₃ independently represents H, C₁₋₃₀ alkyl, —NH₂, —NHC₁₋₃₀ alkyl or —N(C₁₋₃₀ alkyl)₂, where each C₁₋₃₀ alkyl is independently optionally substituted by one or more halogen, —OH, —CN and —NO₂ groups; and R₂, R₄, R₆, R₅, R₁₀ and R₁₂ independently represent H.

In a further aspect, R₁, R₃, R₅, R₇, R₉ and R₁₁ independently represent a compound of formula II, formula III, or formula IV, each R₁₃ independently represents H or C₁₋₃₀ alkyl, where each C₁₋₃₀ alkyl is independently optionally substituted by one or more halogen, —OH, —CN and —NO₂ groups; and R₂, R₄, R₆, R₅, R₁₀ and R₁₂ independently represent H.

In an additional aspect, at least one of R₁, R₃, R₅, R₇, R₉ and R₁₁ represent a compound of formula II, formula III, or formula IV, and each R₁₃ independently represents H or C₁₋₃₀ alkyl. In another aspect, at least one of R₁, R₃, R₅, R₇, R₉ and R₁₁ represent a compound of formula II, formula III or formula IV, and each R₁₃ represents H.

In a particular aspect, the compound is inositol hexaphosphate (IP6). In other aspects, the compound is inositol monophosphate (IP1), inositol diphosphate (IP2), inositol triphosphate (IP3), inositol tetraphosphate (IP4), or inositol pentaphosphate (IP5). In some aspects, the compound comprises a combination of IP1, IP2, IP3, IP4, IP6 and IP6. In some aspects, the IP6 can form other inositol phosphates (IP5, IP4, IP3, IP2, IP1) by dephosphorylation in vivo. Inositol is assumed to mean any isomeric form of the molecule, for example, myoinositol.

In some aspects, the compounds for use in the present invention are those of formula I wherein:

R₇ is OSO₃⁻, and R₉, R₅, R₃, R₁ and R₁ are independently selected from OPO₃²⁻, OPSO₂²⁻ or OSO₃⁻;
R₉, R₅ and R₁ are OPO₃²⁻ and R₇, R₃ and R₁ are OSO₃⁻;
R₉, R₅ and R₁ are OSO₃⁻ and R₇, R₃ and R₁ are OPO₃²⁻;
R₃, R₁ and R₁ are OSO₃⁻ and R₉, R₇ and R₅ are OPO₃²⁻;
R₃, R₁ and R₁ are OPO₃²⁻ and R₉, R₇ and R₅ are OSO₃⁻;
R₇ and R₁ are OPO₃²⁻ and R₉, R₅, R₃, and R₁ are OPO₃²⁻.
R₇ and R₁ are OSO₃⁻ and R₉, R₅, R₃, and R₁ are OPO₃²⁻.
R₇ and R₅ are OPO₃²⁻ and R₉, R₃, R₁, and R₁ are OSO₃⁻; or,
R₇ and R₅ are OSO₃⁻ and R₉, R₃, R₁, and R₁ are OPO₃²⁻.

The inositol phosphates of the present invention also encompass compounds that are produced as metabolites during physiological dephosphorylation (or desulfation or dethiosulfation in the case of compounds comprising sulfate or thiophosphate groups).

In some aspects, the compound administered in a dosage according to the methods disclosed herein is a prodrug that after undergoing hydrolysis or other intracellular or extracellular processing yields an inositol phosphate of the present invention.

The inositol phosphates of the present invention encompass also any combination of the inositol phosphate, inositol phosphate analogs, and derivatives thereof disclosed herein.

All compounds of formula I contain radicals with C—O—P or C—O—S bonds, which provide the compounds with an affinity for calcium-containing crystals and a sufficiently labile bond to be hydrolyzed in vivo, thereby preventing irreversible binding to calcium-containing crystals such as the hydroxyapatite (HAP) in bone, which would have a negative impact on bone remodeling, as is the case with bisphosphonates when administered long term as said compounds contain P—C—P bonds that cannot be hydrolyzed by the body. At the other extreme are phosphorylated compounds that do not contain said C—O—P bonds, such as pyrophosphates, the P—O—P bonds of which mean that they are too readily hydrolyzed in the intestine, thus meaning that only parenteral administration is feasible. The compounds of the present invention, with C O—P bonds, C—O—S bonds, and combinations thereof represent an adequate midpoint due to the efficacy thereof and the fact that the body presents mechanisms for eliminating said compounds, thus reducing the risk of side effects (e.g., compounds with P—C—P bonds can present half-lives of several months which in vivo, thereby affecting, e.g., bone remodeling).

The term “alkyl” or “alkyl group” in the context of the present invention refers to a saturated hydrocarbon moiety, which can be linear, branched, cyclic or cyclic with linear or branched side chains. The term alkyl includes partially unsaturated hydrocarbons such as propenyl. Examples are methyl, ethyl, n- or isobutyl, n- or cyclohexyl. The term alkyl can extend to alkyl groups linked or bridged by hetero atoms. Hetero atoms in the context of the present invention are nitrogen (N), sulfur (S) and oxygen (O).

An “amine function” or “amine group” is a function NR′R″, with R′ and R″ selected independently from hydrogen and C₁-C₅ alkyl. In some aspects, R′ and R″ are selected from hydrogen and C₁-C₃ alkyl. A “hydroxy function” or “hydroxy group” is OH.

A “thiol function” or “thiol group” is SH. A “carboxylic acid function” or “carboxylic acid group” is COOH or its anion, COO—. A “carboxylic amide” is CONR′R″, with R′ and R″ independently having the meanings indicated above. A “sulfonic acid” is SO₃H. A “sulfonic acid amide” is SO₂NR′R″, with R′ and R″ independently having the meanings indicated above.

A “C₁-C₃ alkyl” in the context of the present invention refers to a saturated linear or branched hydrocarbon having 1, 2, or 3 carbon atoms, wherein one carbon-carbon bond can be unsaturated and one CH₂ moiety can be exchanged for oxygen (ether bridge). Non-limiting examples for a C₁-C₃ alkyl are methyl, ethyl, propyl, prop-2-enyl and prop-2-inyl.

A “C₁-C₅ alkyl” in the context of the present invention refers to a saturated linear or branched hydrocarbon having 1, 2, 3, 4 or 5 carbon atoms, wherein one or two carbon-carbon bond can be unsaturated and one CH₂ moiety can be exchanged for oxygen (ether bridge). Non-limiting examples for a C₁-C₅ alkyl include the examples given for C₁-C₃ alkyl above, and additionally n-butyl, 2-methylpropyl, tert-butyl, 3-methylbut-2-enyl, 2-methylbut-3-enyl, 3-methylbut-3-enyl, n-pentyl, 2-methylbutyl, 3-methylbutyl, 1,1-dimethylpropyl, 1,2-dimethylpropyl, 1,2-dimethylpropyl, but-3-enyl, but-3-inyl and pent-4-inyl. A “C₃⁻ C₁₀ alkyl” in the context of the present invention refers to a saturated linear or branched hydrocarbon having 3, 4, 5, 6, 7, 8, 9 or 10 carbon atoms, wherein 1, 2 or 3 carbon-carbon bonds can be unsaturated and one CH₂ moiety can be exchanged for oxygen (ether bridge).

The term “C₁₋₃₀ alkyl” as a group or part of a group, refers to a linear or branched chain alkyl group containing between 1 and 30 carbon atoms including, amongst others, methyl, ethyl, propyl, isopropyl, butyl, isobutyl, sec-butyl, tert-butyl, pentyl, hexyl, decyl and dodecyl groups.

The term “C₂₋₃₀ alkenyl” refers to a linear or branched alkyl chain containing between 2 and 30 carbon atoms and also contains one or more double bonds. Examples include, amongst others, ethenyl, 1-propenyl, 2-propenyl, isopropenyl 1-butenyl, 2-butenyl, 3-butenyl and 1,3-butadienyl.

The term “C₂₋₃₀ alkynyl” refers to a linear or branched alkyl chain containing between 2 and 30 carbon atoms and also contains one or more triple bonds. Examples include, amongst others, ethynyl, 1-propynyl, 2-propynyl, 1-butynyl, 2-butynyl, 3-butynyl and 1,3-butadiynyl.

A “Cy₁ group” refers to a three- to 10-membered carbocyclic or heterocyclic ring that can be saturated, partially unsaturated or aromatic and which is bound to the rest of the molecule via any available C atom. When heterocyclic, Cy₁ contains between one and four heteroatoms selected from amongst N, O and S. Moreover, Cy₁ can optionally be fused with up to four five- or six-membered carbocyclic or heterocyclic rings, which can be saturated, partially unsaturated or aromatic. If the fused ring is a heterocycle, said ring contains one or two heteroatoms selected from amongst N, O and S. Examples of Cy₁ include, amongst others, phenyl, naphthyl, thienyl, furyl, pyrrolyl, thiazolyl, isothiazolyl, imidazolyl, pyrazolyl, 1,2,3-triazolyl, 1,2,4-triazolyl, tetrazolyl, 1,3,4-thiadiazolyl, 1,2,4-thiadiazolyl, pyridyl, pyrazinyl, pyrimidinyl, pyridazinyl, benzimidazolyl, benzofuranyl, isobenzofuranyl, indolyl, isoindolyl, benzothiophenyl, benzothiazolyl, cyclopropyl, cyclobutyl, cyclopentyl, cyclohexyl, cycloheptyl, azetidinyl and aziridinyl.

A “C₁₋₃₀ alkoxy group” as a group or part of a group refers to an OC₁₋₃₀alkyl group, where the C₁₋₃₀alkyl part has the same meaning as above. Examples include methoxy, ethoxy, propoxy, isopropoxy, butoxy, isobutoxy, sec-butoxy and tert-butoxy.

A “C₁₋₃₀ alkylthionyl group” as a group or part of a group refers to an SOC₁₋₃₀alkyl group, where the C₁₋₃₀alkyl part has the same meaning as above. Examples include methylthionyl, ethylthionyl, propyithionyl, isopropyithionyl, butylthionyl, isobutyithionyl, sec-butylthionyl and tert-butylthionyl.

A “C₁₋₃₀ acyloxy group” as a group or part of a group refers to a COC₁₋₃₀alkyl group, where the C₁₋₃₀alkyl part has the same meaning as above. Examples include acetyl, ethanoyl, propanoyl and 2,2-diisopropylpentanoyl.

A “halogen radical” or the halo abbreviation thereof refers to fluorine, chlorine, bromine and iodine.

A “trihalo C₁₋₃₀ alkyl group” refers to a group resulting from the substitution of three hydrogen atoms of a C₁₋₃₀alkyl group by three halogen radicals as defined above. Examples include, amongst others, trifluoromethyl, tribromomethyl, trichloromethyl, triiodomethyl, trifluoroethyl, tribromoethyl, trichloroethyl, triiodoethyl, tribromopropyl, trichloropropyl and triiodopropyl.

An “—NHC₁₋₃₀ alkyl group” refers to a group resulting from the substitution of one hydrogen atom of an —NH₂ group by a C₁₋₃₀ alkyl group as defined above. Examples include, amongst others, methylamine, ethylamine, propylamine, butylamine and pentylamine.

A “—N(C₁₋₃₀ alkyl)₂ group” refers to a group resulting from the substitution of two hydrogen atoms of an —NH₂ group by a C₁₋₃₀ alkyl group as defined above. Examples include, amongst others, dimethylamine, diethylamine, diisopropylamine, dibutylamine and diisobutylamine.

The expression “optionally substituted by one or more” signifies the possibility that a group can be substituted by one or more (e.g., by 1, 2, 3 or 4) substituents. In some aspects, a group can be substituted by 1, 2 or 3 substituents and even by 1 or 2 substituents provided that the group has sufficient positions that can be substituted available. If present, the substituents can be the same or different and can be located at any available position.

In some aspects, the inositol phosphates of the present invention comprise the compounds disclosed in WO2017098033 and WO2017098047, and US U.S. Pat. No. 9,358,243. In some aspects, the inositol phosphates of the present invention used comprise the compounds disclosed in FIGS. 1-6.

In some aspects, the inositol phosphates, inositol phosphate analogs, and derivatives thereof, comprise compounds of formula (VIII), formula (IX), or formula (X):

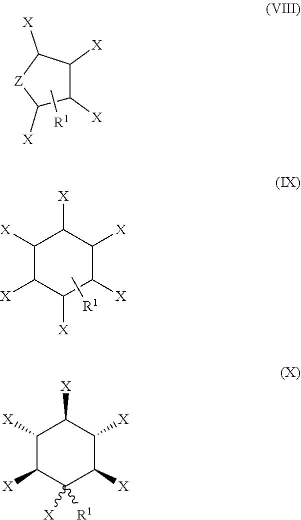

wherein each X independently is selected from OPO₃²⁻, OPSO₂²⁻, or OSO₃; Z is an alkyl chain comprising 1 to 3 carbon and/or hetero atoms, optionally comprising a group X, wherein X is also selected from OPO₃²⁻, OPSO₂²⁻, or OSO₃⁻; and, R¹ is an optional heterologous moiety (See section 2.2. below). In some aspects, the molecule comprises more than one heterologous moiety, in which case the heterologous moieties can be the same or be different.

In some aspects, Z, as used in formula (VIII), is CH₂, CHX, CHR¹, CXR¹, CH₂—CH₂, CH₂—CHX, CHX—CHX, CHR¹—CHX, CXR¹—CHX, CHR¹—CH₂, CXR¹CH₂, CHR¹—CHOH, CH₂—CH₂—CH₂, CH₂—O—CH₂, CHOH—CH₂—CH₂, CHOH—CHOH—CHR¹, CHOH—CHR¹—CHOH, CHX—CH₂—CH₂, CH₂—CHX—CH₂, CHX—CHX—CH₂, CHX—CH₂—CHX or CHX—CHR¹—CHX, wherein X independently is selected from OPO₃²⁻, OPSO₂²⁻, and OSO₃⁻.

In some aspects, Z, as used in formula (VIII), is (CHX)ₚCHX(CHX)_{q}; wherein p and q each independently from the other have a value from 0 to 2, with the proviso that (p+q) has a value of 0, 1 or 2; one or two or three X can be a heterologous moiety (e.g., PEG) and the remaining X are independently selected from OPO₃²⁻, OPSO₂ ²⁻, and OSO₃⁻. In some aspects, not all X of Z are PO₃²⁻. In some aspects, not all X of Z are OSO₃⁻.

In some aspects, one, two, or three of the X in compounds of formula (VIII), formula (IX), or formula (X) can be heterologous moiety and the remaining X can independently be selected from OPO₃²⁻, OPSO₂²⁻, or OSO₃.

Formula (VII) above describes a five-membered, six-membered, or seven-membered alkyl ring, and the optional heterologous moiety or moieties is/are attached to one of the carbon atoms forming the ring.

In some aspects, the inositol phosphates, inositol phosphate analogs, and derivatives thereof used, e.g., in the methods and compositions disclosed herein, comprise compounds of formula (XI) or formula (XII):

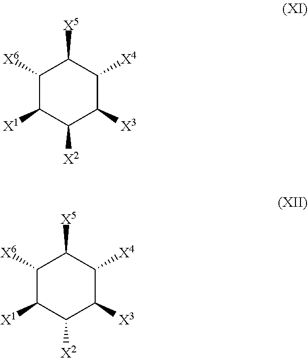

wherein:
X² is OSO₃⁻, and X¹, X³, X⁴, X⁵ and X⁶ are independently selected from OPO₃²⁻, OPSO₂²⁻ or OSO₃⁻;
X¹, X³ and X⁵ are OPO₃²⁻ and X², X⁴ and X⁶ are OSO₃⁻;
X¹, X³ and X⁵ are OSO₃⁻ and X², X⁴ and X⁶ are OPO₃²⁻;
X⁴, X⁵ and X⁶ are OSO₃⁻ and X¹, X² and X³ are OPO₃²⁻;
X⁴, X⁵ and X⁶ are OPO₃²⁻ and X¹, X² and X³ are OSO₃⁻;
X² and X⁵ are OPO₃²⁻ and X¹, X³, X⁴, and X⁶ are OPO₃²⁻;
X² and X⁵ are OSO₃⁻ and X¹, X³, X⁴, and X⁶ are OPO₃²⁻;
X² and X³ are OPO₃²⁻ and X¹, X⁴, X⁵, and X⁶ are OSO₃⁻; or,
X² and X³ are OSO₃⁻ and X¹, X⁴, X⁵, and X⁶ are OPO₃²⁻

In some aspects, the inositol phosphates of the present invention or metabolites thereof can be detected and/or quantified using the methods disclosed in U.S. Pat. No. 9,612,250. See also, U.S. Pat. Nos. 8,377,909, 8,778,912, and US20070066574.

The compounds disclosed herein can be present in any form commonly used in pharmaceutical technology. Particular aspects include, but are not limited to, the sodium salt, magnesium salt, potassium salt, ammonium salt, free acid, or a mixture of the preceding forms. Other pharmaceutically acceptable salts are known to the skilled artisan and can be readily obtained. In a particular aspect, the compound for use as defined in the first aspect of the invention is a sodium salt, for example, inositol hexaphosphate hexasodium.

The present invention also contemplates sodium salts of inositol monophosphate, inositol diphosphate, inositol triphosphate, inositol tetraphosphate and inositol pentaphosphate in any of inositol isomeric forms, in particular, myo-inositol. A particular example of the compounds for use in the present invention is myo-inositol hexaphosphate hexasodium salt. Sodium salts provide several advantages in terms of the manufacturing and the level of impurities of the resulting IP6 formulations.

### 2.2. Heterologous Moiety

In some aspects, the present invention refers to a compound of general formula I, as defined above, wherein the heterologous moiety is selected from a radical of formula V, a radical of formula VI and a radical of formula VII:

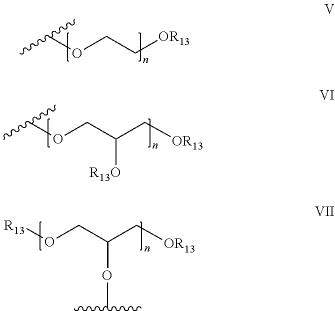

and wherein n is an integer in the range from 2 to 200, and R₁₃ is selected from H, methyl or ethyl.

In some aspects, compounds for use in the present invention, for example, the inositol phosphate derivatives of the present invention, can comprise one or two radicals selected from the radicals of formulas V, VI and VII. These radicals are heterologous moieties conferring an advantageous property with respect to a corresponding molecule lacking such heterologous moiety or moieties. Examples of said advantageous properties that can be conferred by a heterologous moiety or a combination thereof to an inositol phosphate or inositol phosphate analogs include, but are not limited to (a) an increase in solubility, (b) a decrease in degradation or metabolization rate, (c) an increase in plasma half-life, (d) a decrease in liver metabolization rate (e) a decrease in clearance rate, (f) a decrease of toxicity, (g) a decrease of irritability and (h) reduced side effects among others. These advantageous properties can be evaluated or quantified using methods known in the art without undue experimentation.

In some aspects, the heterologous moiety is, for instance, a polyethylene glycol (PEG) or a polyglycerol (PG). Thus, in certain aspects, the compound for use in the invention is any of the compounds as defined in the aspects disclosed above comprising a heterologous moiety, that is, one of the radicals of formula I is selected from the radicals of formulas V, VI and VII. In some aspects the heterologous moiety comprises a polyethylene glycol (PEG). In certain aspects the heterologous moiety consists of polyethylene glycol, that is to say, at least one of R₁, R₃, R₅, R₇, R₉ and R₁₁ of the compound of formula I according to the first aspect of the invention is a radical of formula V. Alternatively, the heterologous moiety comprises a polyglycerol. In certain aspects the heterologous moiety consists on polyglycerol, that is to say, at least one of R₁, R₃, R₅, R₇, R₉ and R₁₁ of the compound of formula I according to the first aspect of the invention is selected from a radical of formula VI or VII. In other aspects the compound of formula I according to the first aspect of the invention contains one, two or three radicals selected from a radical of formula VI or VII, for example two PEGs (radical of formula V), Three PEGs, two polyglycerols (radical of formula VI), three PGs, or any combinations thereof, for example, one PEG and one PG, or two PEGs and one polyglycerol. In certain aspects all of the remaining radicals of formula I (i.e., those that are not a radical selected from V, VI and VII) are a radical selected from II, III and IV. In some aspects the compound of formula I according to the first aspect of the invention contains two radicals selected from a radical of formula VI or VII, for example two PEGs (radical of formula V) or two polyglycerols (radical of formula VI) or one PEG and one polyglycerol and the remaining radicals are all a radical of formula II. In some aspects R₃ and R₇ of the compound of formula I are selected from a radical of formula V, VI and VII. In some aspects, R₃ and R₇ of the compound of formula I are radicals of formula V, and R₁, R₅, R₉ and R₁₁ of the compound of formula I are radicals of formula II.

The radicals of formulas V, VI and VII have R₁₃═H, methyl or ethyl and n is an integer from 2 to 200. In some aspects R₁₃═H. In particular aspects n is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200.

In some aspects, n is between 2 and 10, between 10 and 20, between 20 and 30, between 30 and 40, between 40 and 50, between 50 and 60, between 60 and 70, between 70 and 80, between 80 and 90, between 90 and 100, between 100 and 110, between 110 and 120, between 120 and 130, between 130 and 140, between 140 and 150, between 150 and 160, between 160 and 170, between 170 and 180, between 180 and 190, or between 190 and 200.

In some specific aspects, n has a value from 2 to 200, from 2 to 20, from 10 to 30, or from 9 to 45.

In some aspects, the PEG is a branched PEG. Branched PEGs have three to ten PEG chains emanating from a central core group.

In certain aspects, the PEG moiety is a monodisperse polyethylene glycol. In the context of the present invention, a monodisperse polyethylene glycol (mdPEG) is a PEG that has a single, defined chain length and molecular weight. mdPEGs are typically generated by separation from the polymerization mixture by chromatography. In certain formulae, a monodisperse PEG moiety is assigned the abbreviation mdPEG. In some aspects, the PEG is a Star PEG. Star PEGs have 10 to 100 PEG chains emanating from a central core group.

In some aspects, the PEG is a Comb PEGs. Comb PEGs have multiple PEG chains normally grafted onto a polymer backbone.

In certain aspects, the PEG has a molar mass between 100 g/mol and 3000 g/mol, particularly between 100 g/mol and 2500 g/mol, more particularly of approx. 100 g/mol to 2000 g/mol. In certain aspects, the PEG has a molar mass between 200 g/mol and 3000 g/mol, particularly between 300 g/mol and 2500 g/mol, more particularly of approx. 400 g/mol to 2000 g/mol.

In some aspects, the PEG is PEG₁₀₀, PEG₂₀₀, PEG₃₀₀, PEG₄₀₀, PEG₅₀₀, PEG₆₀₀, PEG₇₀₀, PEG₅₀₀, PEG₉₀₀, PEG₁₀₀₀, PEG₁₁₀₀, PEG₁₂₀₀, PEG₁₃₀₀, PEG₁₄₀₀, PEG₁₅₀₀, PEG₁₆₀₀, PEG₁₇₀₀, PEG₁₅₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₂₆₀₀, PEG₂₇₀₀, PEG₂₅₀₀, PEG₂₉₀₀, or PEG₃₀₀₀. In one particular aspect, the PEG is PEG₄₀₀. In another particular aspect, the PEG is PEG₂₀₀₀.

In other particular aspects R₃ and/or R₇ of the compound of formula I is a radical of formula V where R₁₃ is H and n is an integer from 9 to 45. In other particular aspects R₃ and R₇ of the compound of formula I is a radical of formula V where R₁₃ is H and n is an integer from 9 to 45 and R₁, R₅, R₉ and R₁₁ are all a radical of formula II. In other particular aspects the compound of formula I is a sodium salt with R₃ and R₇=radical of formula V where R₁₃ is H and n is an integer from 9 to 45, and R₁, R₅, R₉ and R₁ are all a radical of formula II.

In some other aspects, the heterologous moiety is a polyglycerol (PG) described by the formula ((R₃—O—(CH₂—CHOH—CH₂O)ₙ—) with R₃ being hydrogen, methyl or ethyl, and n having a value from 3 to 200. In some aspects, n has a value from 3 to 20. In some aspects, n has a value from 10 to 30. In some alternatives of these aspects, n has a value from 9 to 45. In some aspects, the heterologous moiety is a branched polyglycerol described by the formula (R³—O—(CH₂—CHOR⁵—CH₂—O)ₙ—) with R⁵ being hydrogen or a linear glycerol chain described by the formula (R³—O—(CH₂—CHOH—CH₂—O)ₙ—) and R³ being hydrogen, methyl or ethyl. In some aspects, the heterologous moiety is a hyperbranched polyglycerol described by the formula (R³—O—(CH₂—CHOR⁵—CH₂—O)ₙ—) with R₅ being hydrogen or a glycerol chain described by the formula (R³—O—(CH₂—CHOR⁶—CH₂—O)ₙ—), with R⁶ being hydrogen or a glycerol chain described by the formula (R³—O—(CH₂—CHOR⁷—CH₂—O)ₙ—), with R⁷ being hydrogen or a linear glycerol chain described by the formula (R³—O—(CH₂—CHOH—CH₂—O)ₙ—) and R³ being hydrogen, methyl or ethyl. Hyperbranched glycerol and methods for its synthesis are known in the art. See Oudshorn M, et al., Biomaterials 2006; 27:5471-5479, Wilms D, et al., Acc Chem Res 2010; 43:129-141 and references cited therein.

In certain aspects, the PG has a molar mass between 100 g/mol and 3000 g/mol, particularly between 100 g/mol and 2500 g/mol, more particularly of approx. 100 g/mol to 2000 g/mol. In certain aspects, the PG has a molar mass between 200 g/mol and 3000 g/mol, particularly between 300 g/mol and 2500 g/mol, more particularly of approx. 400 g/mol to 2000 g/mol.

In some aspects, the PG is PG₁₀₀, PG₂₀₀, PG₃₀₀, PG₄₀₀, PG₅₀₀, PG₆₀₀, PG₇₀₀, PG₅₀₀, PG₉₀₀, PG₁₀₀₀, PG₁₁₀₀, PG₁₂₀₀, PG₁₃₀₀, PG₁₄₀₀, PG₁₅₀₀, PG₁₆₀₀, PG₁₇₀₀, PG₁₈₀₀, PG₁₉₀₀, PG₂₀₀₀, PG₂₁₀₀, PG₂₂₀₀, PG₂₃₀₀, PG₂₄₀₀, PG₂₅₀₀, PG₁₆₀₀, PG₁₇₀₀, PG₁₈₀₀, PG₁₉₀₀, PG₂₀₀₀, PG₂₁₀₀, PG₂₂₀₀, PG₂₃₀₀, PG₂₄₀₀, PG₂₅₀₀, PG₂₆₀₀, PG₂₇₀₀, PG₂₅₀₀, PG₂₉₀₀, or PG₃₀₀₀. In one particular aspect, the PG is PG₄₀₀. In another particular aspect, the PG is PG₂₀₀₀.

In other particular aspects R₃ and/or R₇ of the compound of formula I is a radical of formula VI where R₁₃ is H and n is an integer from 9 to 45. In other particular aspects R₃ and R₇ of the compound of formula I is a radical of formula VI where R₁₃ is H and n is an integer from 9 to 45 and R₁, R₅, R₉ and R₁₁ are all a radical of formula II. In other particular aspects the compound of formula I is a sodium salt with R₃ and R₇=radical of formula VI where R₁₃ is H and n is an integer from 9 to 45, and R₁, R₅, R₉ and R₁ are all a radical of formula II.

### 3. Pharmaceutical Compositions

In another aspect, the present invention also refers to pharmaceutical compositions comprising a compound, as defined in any of the aspects disclosed above. In some aspects, the pharmaceutical composition comprises a compound, as defined in any of the aspects disclosed above, together with one or more pharmaceutically acceptable excipients or carriers. These pharmaceutical compositions are for use in increasing tissular perfusion and/or oxygenation in a subject in need thereof. In some aspects, these pharmaceutical compositions are for use in the treatment or prevention of ischemia and/or an ischemia-related disease or condition. In some aspects, the pharmaceutical compositions of the present invention are used for the treatment or prevention of PAD or CLI.

The term “excipient” as used herein refers to a substance which helps absorption of the elements of the pharmaceutical composition, stabilizes said elements, activates or helps preparation of the composition. Thus, examples of excipients used in parenteral formulations include, but are not limited to, antimicrobial agents (e.g., benzalkonium chloride, metacresol, thimerosal), co-solvents (e.g., ethanol), buffers and pH adjusting factors (e.g., carbonate, citrate, phosphate solutions).

As is the case for the excipient, the “pharmaceutically acceptable vehicle” is a substance used in the composition to dilute any of the components contained therein to a determined volume or weight. The pharmaceutically acceptable vehicle is an inert substance or a substance with an analogous action to any of the elements comprising the pharmaceutical composition of the present invention. The role of said vehicle is to allow the incorporation of other elements, allow better dosing and administration or to provide consistency and shape to the composition.

Pharmaceutical compositions can comprise from approximately 1% to approximately 95% of the compound as defined in any of the aspects disclosed above. In some aspects, the pharmaceutical compositions of the present invention can comprise, for instance, from approximately 20% to approximately 90%, or from 20% to 80%, or from 20% to 70%, or from 20% to 60%, or from 20% to 50%, or from 30% to 90%, or from 40% to 90%, or from 50% to 90%, or from 60% to 90%, or from 30% to 70% of the compound as defined in any of the aspects disclosed above.

In some aspects, the concentration of inositol phosphate of the present invention (e.g., myo-inositol hexaphosphate or an analog or derivative thereof, or a combination thereof) in each dose of the pharmaceutical composition is between about 12.5 mM and about 135 mM. In some versions of this aspect, the concentration of inositol phosphate of the present invention (e.g., myo-inositol hexaphosphate or an analog or derivative thereof, or a combination thereof) in each dose of the pharmaceutical composition is about 25 mM, about 39 mM or about 114 mM.

Formulations of a pharmaceutical composition suitable for parenteral administration comprise a compound as defined in any of the aspects disclosed above mixed with a pharmaceutically acceptable carrier (e.g., as sterile water or sterile isotonic saline solution). Such formulations can be prepared, packaged, or sold in a form suitable for bolus administration or for continuous administration. Injectable formulations can be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations can further comprise one or more additional ingredients including, but not limited to, suspending, stabilizing, or dispersing agents.

In some aspects, in a formulation for parenteral administration, the active agent (e.g., a compound as defined in any of the aspects disclosed above) is provided in dry (i.e., powder or granular) form for reconstitution with a suitable vehicle (e.g., sterile pyrogen-free water) prior to parenteral administration of the reconstituted composition.

The pharmaceutical compositions can be prepared, packaged, or sold in the form of a sterile injectable aqueous or oily suspension or solution. This suspension or solution can be formulated according to the known art, and may comprise, in addition to the active agent (e.g., compound as defined in any of the aspects disclosed above), additional ingredients such as the dispersing agents, wetting agents, or suspending agents described herein. Such sterile injectable formulations can be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides.

Other parentally-administrable formulations which are useful include those which comprise the active agent (e.g., compound as defined in any of the aspects disclosed above) in microcrystalline form, in a liposomal preparation, or as a component of a biodegradable polymer system.

Compositions for sustained release or implantation can comprise pharmaceutically acceptable polymeric or hydrophobic materials such as an emulsion, an ion exchange resin, a sparingly soluble polymer, or a sparingly soluble salt.

Controlled- or sustained-release formulations of a pharmaceutical composition of the present invention can be made using conventional technology. In some cases, the dosage forms to be used can be provided as slow or controlled-release of one or more active agents therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known in the art, including those described herein, can be readily selected for use with the pharmaceutical compositions of the invention. Thus, single unit dosage forms suitable for parenteral or topical administration, such as injectable solutions, gels, creams, and ointments, which are adapted for controlled-release are encompassed by the present invention.

Most controlled-release pharmaceutical products have a common goal of improving therapy over that achieved by their non-controlled counterparts. Ideally, the use of an optimally designed controlled-release preparation in medical treatment is characterized by a minimum of therapeutic agent being employed to cure or control the condition in a minimum amount of time. Advantages of controlled-release formulations include extended activity of the therapeutic agent, reduced dosage frequency, and increased patient compliance. In addition, controlled-release formulations can be used to affect the time of onset of action or other characteristics, such as blood level of the therapeutic agent, and thus can affect the occurrence of side effects.

Most controlled-release formulations are designed to initially release an amount of therapeutic agent that promptly produces the desired therapeutic effect, and gradually and continually release of other amounts of therapeutic agent to maintain this level of therapeutic effect over an extended period of time. In order to maintain this constant level of therapeutic agent in the body, the therapeutic agent must be released from the dosage form at a rate that will replace the amount of therapeutic agent being metabolized and excreted from the body.

Controlled-release of an active agent can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active agent.

In certain aspects, the formulations of the present invention can be, but are not limited to, short-term, rapid-offset, as well as controlled, for example, sustained release, delayed release and pulsatile release formulations.

The term sustained release is used in its conventional sense to refer to therapeutic agent formulation (e.g., compound as defined in any of the aspects disclosed above)) that provides for gradual release of a therapeutic active agent over an extended period of time, and that can, although not necessarily, result in substantially constant blood levels of a therapeutic agent over an extended time period. The period of time can be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the present invention can be administered in the form of microparticles, for example, by injection or in the form of wafers or discs by implantation. In certain aspects, the compounds of the invention are administered to a patient, alone or in combination with another pharmaceutical agent, using a sustained release formulation.

The term delayed release is used herein in its conventional sense to refer to a therapeutic agent formulation that provides for an initial release of the therapeutic agent after some delay following therapeutic agent administration. The delay may be from about 10 minutes up to about 12 hours. The term pulsatile release is used herein in its conventional sense to refer to a therapeutic agent formulation that provides release of the therapeutic agent in such a way as to produce pulsed plasma profiles of the therapeutic agent after administration. The term immediate release is used in its conventional sense to refer to a therapeutic agent formulation that provides for release of the therapeutic agent immediately after administration.

Additional formulations and dosage forms of the compositions of the present invention include dosage forms as described in U.S. Pat. Nos. 6,340,475, 6,488,962, 6,451,808, 5,972,389, 5,582,837, and 5,007,790; US20030147952, 20030104062, 20030104053, 20030044466, 20030039688, and 20020051820; WO 2003035041, WO2003035040, WO2003035029, WO200335177, WO2003035039, WO2002096404, WO2002032416, WO2001097783, WO2001056544, WO2001032217, WO1998055107, WO1998011879, WO1997047285, WO1993018755, and WO1990011757.

Medicaments according to the invention are manufactured by methods known in the art, especially by conventional mixing, coating, granulating, dissolving or lyophilizing.

The present invention also provides a compound, a combination of compounds, or pharmaceutical formulation as defined in any of the above aspects of the invention, in the broadest definition given, or as specified in any of the aspects presented above, for use as a medicament.

### 4. Methods and Routes of Administration

In some aspects, the compound, pharmaceutical composition or combined preparation as defined in any of the aspects disclosed above is administered jointly, concurrently or sequentially with another therapeutic agent. In some versions of this aspect, the additional therapeutic agent comprises cilostazol, pentoxifylline or combination thereof.

In some aspects, the administration of an effective amount of compound, pharmaceutical composition or combined preparation as defined in any of the aspects above is provided. Said compound, pharmaceutical composition or combined preparation can be administered parenterally such as, for example, intravenously, intraperitoneally, intramuscularly, intra-arterially, intradermal, intrathecal, epidural or spinal or subcutaneously. The parenteral administration may be by bolus injection or by intravenous infusion.

In a particular aspect of the present invention, myo-inositol hexaphosphate (or a formulation comprising myo-inositol hexaphosphate such as SNF472) is administered via intravenous infusion. In another particular aspect of the present invention, myo-inositol hexaphosphate is administered subcutaneously. In another aspect a derivative of inositol or myo-inositol hexaphosphate derivative, for example, a compound of formula I with R₃ and R₇=radical of formula V where R₁₃ is H and n is an integer from 2 to 200, and R₁, R₅, R₉ and R₁₁ are all a radical of formula II (or a sodium salt thereof) is administered via intravenous infusion. In another aspect, a derivative of inositol or myo-inositol hexaphosphate derivative, for example, a compound of formula I with R₃ and R₇=radical of formula V where R₁₃ is H and n is an integer from 2 to 200, and R₁, R₅, R₉ and R₁₁ are all a radical of formula II (or a sodium salt thereof) is administered subcutaneously.

Alternatively, the compound, pharmaceutical composition or combined preparation can be administered as a component of a hemodialysis, hemofiltration, or peritoneal dialysis solution or system.

In the particular case of patients treated with dialysis, a very appropriate method of administration consists of an administration (e.g., a non-bolus type administration) of an inositol phosphate of the present invention via the dialysis apparatus (before or after the filter) instead of directly injecting the inositol phosphate of the present invention into the patient intravenously. Thus, blood can be treated with the inositol phosphate of the present invention (e.g., myo-inositol hexaphosphate) as it leaves the patient and circulates through the dialysis circuit and, when the blood containing the inositol phosphate of the present invention returns to the body.

Thus, in some aspects, the compound, pharmaceutical composition or combined preparation, as defined in any of the aspects disclosed above, is administered to a patient during hemodialysis. In some aspects, the compound, pharmaceutical composition or combined preparation, as defined in any of the aspects disclosed above, is administered to the blood extracted from the patient during hemodialysis, preferably before it is filtered (i.e., the therapeutic agent is administered to the patient's unfiltered blood in the dialysis circuit). In some aspects, the compound is inositol hexaphosphate, in particular myo-inositol hexaphosphate sodium salt or a derivative thereof, namely, a compound of formula I with R₃ and R₇=radical of formula V where R₁₁ is H and n is an integer from 2 to 200, and R₁, R₅, R₉ and R₁₁ are all a radical of formula II (or a sodium salt thereof).

In the case of dialysis patients, administration of an inositol phosphate of the present invention (e.g., myo-inositol hexaphosphate) via the dialysis apparatus allows the blood to equilibrate with the dialysis fluid prior to returning to the body; thus, although inositol phosphate of the present invention (e.g., myo-inositol hexaphosphate) can sequester ionic calcium, this fact is compensated when the blood passes through the dialysis filter thereby eliminating said side effect and significantly improving the safety profile. Additionally, administering the inositol phosphate of the present invention (e.g., myo-inositol hexaphosphate) concomitant to hemodialysis, in particular when administered to the unfiltered blood extracted from the patient during hemodialysis, allows for reducing the dose of the compound with consequent advantages in terms of reduced toxicity and minimizing adverse side effects.

In some aspects, the compound, pharmaceutical composition or combined preparation as defined in any of the aspects disclosed above is administered to a patient that is being treated with hemodialysis before the dialysis treatment or after a dialysis treatment.

In general, an effective dose of an inositol phosphate of the present invention (e.g., myo-inositol hexaphosphate) administered according to the methods disclosed herein will depend, for example, on the relative efficacy of the compound concerned, the severity of the disorder treated, and the species and weight of the subject. In some aspects, the effective dose of an inositol phosphate of the present invention for a subject of a certain species (e.g., human) can be calculated based on the experimental data available for a different or reference species (e.g., rat).

Thus, for example, a dose of inositol myo-hexaphosphate administered as part of a regimen comprising the administration of a dosage of 20 mg/kg to a rat subject would be equivalent to administering the same active agent at a dosage of 4.2 mg/kg to a human subject (i.e., a total dose of 300 mg of inositol myo-hexaphosphate to a human subject weighing approximately 70 kg). Likewise, a dosage of 40 mg/kg to a rat subject would be equivalent to administering inositol myo-hexaphosphate at a dosage of 8.4 mg/kg to a human subject as defined before. The dosages can be adjusted based on the subjects age, species, weight, body surface, renal clearance, sex, pathological state, route of administration, concurrent administration of one or more other drugs, and a wide variety of physiologic and psychological factors using methods known in the art (Pan S., et al., Patient Prefer Adherence 2016; 10:549-560; Pai M, Pharmacotherapy 2012; 32:856-868; Hacker M., et al., Eds, “Pharmacology: Principles and Practice” (Academic Press; Burlington, Mass., USA, 2009). The term “mg/kg” as used herein refers to mg of an inositol phosphate of the present invention per kilogram of the body mass (body weight) of the subject.

In some aspects, the dose of inositol phosphate of the present invention (e.g., myo-inositol hexaphosphate) comprises from about 0.001 mg/kg to about 60 mg/kg of an inositol phosphate, an inositol phosphate analog, an inositol phosphate derivative, or combination thereof according the present invention. In some further aspects, the dose of inositol phosphate of the present invention (e.g., myo-inositol hexaphosphate) is between about 0.001 mg/kg and about 20.0 mg/kg, between about 20.0 mg/kg and about 40.0 mg/kg, or between about 40.0 mg/kg and about 60.0 mg/kg.

In some aspects, the dose of inositol phosphate of the present invention (e.g., myo-inositol hexaphosphate) is between about 0.001 mg/kg and about 1.0 mg/kg, between about 1.0 mg/kg and about 10.0 mg/kg, between about 10.0 mg/kg and about 20.0 mg/kg, between about 20.0 mg/kg and about 30.0 mg/kg, between about 30.0 mg/kg and about 40.0 mg/kg, between about 40.0 mg/kg and about 50.0 mg/kg, or between about 50.0 mg/kg and about 60.0 mg/kg.

In some aspects, the dose of inositol phosphate of the present invention (e.g., myo-inositol hexaphosphate) is between about 0.001 mg/kg and about 0.5 mg/kg, between about 0.5 mg/kg and about 1.0 mg/kg, between about 1.0 mg/kg and about 5.0 mg/kg, between about 5.0 mg/kg and about 10.0 mg/kg, between about 10.0 mg/kg and about 15.0 mg/kg, between about 15.0 mg/kg and about 20.0 mg/kg, between about 20.0 mg/kg and about 25.0 mg/kg, between about 25.0 mg/kg and about 30.0 mg/kg, between about 30.0 mg/kg and about 35.0 mg/kg, between about 35.0 mg/kg and about 40.0 mg/kg, between about 40.0 mg/kg and about 45.0 mg/kg, or between about 45.0 mg/kg and about 50.0 mg/kg.

In some aspects, the dose of inositol phosphate of the present invention (e.g., myo-inositol hexaphosphate) is between about 0.001 mg/kg and about 0.25 mg/kg, between about 0.25 mg/kg and about 0.5 mg/kg, between about 0.5 mg/kg and about 0.75 mg/kg, between about 0.75 mg/kg and about 1.0 mg/kg, between about 1.0 mg/kg and about 2.50 mg/kg, between about 2.50 mg/kg and about 5.0 mg/kg, between about 5.0 mg/kg and about 7.5 mg/kg, between about 7.5 mg/kg and about 10.0 mg/kg, between about 10.0 mg/kg and about 12.5 mg/kg, between about 12.5 mg/kg and about 15.0 mg/kg, between about 15.0 mg/kg and about 17.5 mg/kg, between about 17.5 mg/kg and about 20.0 mg/kg, between about 20.0 mg/kg and about 22.5 mg/kg, between about 22.5 mg/kg and about 25.0 mg/kg, between about 25.0 mg/kg and about 27.5 mg/kg, between about 27.5 mg/kg and about 30.0 mg/kg, between about 30.0 mg/kg and about 32.5 mg/kg, between about 32.5 mg/kg and about 35.0 mg/kg, between about 35.0 mg/kg and about 37.5 mg/kg, between about 37.5 mg/kg and about 40.0 mg/kg, between about 40.0 mg/kg and about 42.5 mg/kg, between about 42.5 mg/kg and about 45.0 mg/kg, between about 45.0 mg/kg and about 47.5 mg/kg, between about 47.5 mg/kg and about 50.0 mg/kg, between about 50.0 mg/kg and about 52.5 mg/kg, between about 52.5 mg/kg and about 55.0 mg/kg, between about 55.0 mg/kg and about 57.5 mg/kg, or between about 57.5 mg/kg and about 60.0 mg/kg.

In some aspects, the dose of inositol phosphate of the present invention (e.g., myo-inositol hexaphosphate) is between about 0.25 mg/kg and about 60.0 mg/kg, between about 0.5 mg/kg and about 60.0 mg/kg, between about 0.75 mg/kg and about 60.0 mg/kg, between about 1.0 mg/kg and about 60.0 mg/kg, between about 2.50 mg/kg and about 60.0 mg/kg, between about 5.0 mg/kg and about 60.0 mg/kg, between about 7.5 mg/kg and about 60.0 mg/kg, between about 10.0 mg/kg and about 60.0 mg/kg, between about 12.5 mg/kg and about 60.0 mg/kg, between about 15.0 mg/kg and about 60.0 mg/kg, between about 17.5 mg/kg and about 60.0 mg/kg, between about 20.0 mg/kg and about 60.0 mg/kg, between about 22.5 mg/kg and about 60.0 mg/kg, between about 25.0 mg/kg and about 60.0 mg/kg, between about 27.5 mg/kg and about 60.0 mg/kg, between about 30.0 mg/kg and about 60.0 mg/kg, between about 32.5 mg/kg and about 60.0 mg/kg, between about 35.0 mg/kg and about 60.0 mg/kg, between about 37.5 mg/kg and about 60.0 mg/kg, between about 40.0 mg/kg and about 60.0 mg/kg, between about 42.5 mg/kg and about 60.0 mg/kg, between about 45.0 mg/kg and about 60.0 mg/kg, between about 47.5 mg/kg and about 60.0 mg/kg, between about 50.0 mg/kg and about 60.0 mg/kg, between about 52.5 mg/kg and about 60.0 mg/kg, between about 55.0 mg/kg and about 60.0 mg/kg, or between about 57.5 mg/kg and about 60.0 mg/kg.

In some aspects, the dose of inositol phosphate of the present invention (e.g., myo-inositol hexaphosphate) is between about 0.001 mg/kg and about 57.5 mg/kg, between about 0.001 mg/kg and about 55.0 mg/kg, between about 0.001 mg/kg and about 52.5 mg/kg, between about 0.001 mg/kg and about 50.0 mg/kg, between about 0.001 mg/kg and about 47.5 mg/kg, between about 0.001 mg/kg and about 45.0 mg/kg, between about 0.001 mg/kg and about 42.5 mg/kg, between about 0.001 mg/kg and about 40.0 mg/kg, between about 0.001 mg/kg and about 37.5 mg/kg, between about 0.001 mg/kg and about 35.0 mg/kg, between about 0.001 mg/kg and about 32.5 mg/kg, between about 0.001 mg/kg and about 30.0 mg/kg, between about 0.001 mg/kg and about 27.5 mg/kg, between about 0.001 mg/kg and about 25.0 mg/kg, between about 0.001 mg/kg and about 22.5 mg/kg, between about 0.001 mg/kg and about 20.0 mg/kg, between about 0.001 mg/kg and about 27.5 mg/kg, between about 0.001 mg/kg and about 25.0 mg/kg, between about 0.001 mg/kg and about 22.5 mg/kg, between about 0.001 mg/kg and about 20.0 mg/kg, between about 0.001 mg/kg and about 17.5 mg/kg, between about 0.001 mg/kg and about 15.0 mg/kg, between about 0.001 mg/kg and about 12.5 mg/kg, between about 0.001 mg/kg and about 10.0 mg/kg, between about 0.001 mg/kg and about 7.5 mg/kg, between about 0.001 mg/kg and about 5.0 mg/kg, or between about 0.001 mg/kg and about 2.5 mg/kg.

### 5. Indications

The compounds, pharmaceutical compositions, combined preparations, methods and routes of administration, as defined in any of the aspects disclosed above, can be used for increasing tissular perfusion and/or oxygenation in a subject in need thereof.

The terms “ischemia-related disease or condition” as used herein refer to any diseases or condition related to or arising from an ischemic event or injury. Examples of ischemia-related diseases or conditions include, but are not limited, to cerebrovascular (e.g., stroke, transient ischemic attack (TIA), subarachnoid hemorrhage, vascular dementia), cardiovascular (e.g., myocardial infarction, angina pectoris), gastrointestinal (e.g., colitis), peripheral (e.g., acute limb ischemia) and cutaneous (e.g., cyanosis, gangrene) diseases or conditions.

In some aspects, the compounds, pharmaceutical compositions, combined preparations, methods and routes of administration of the present invention can be used for the treatment or prevention of ischemia and/or an ischemia-related disease or condition in a subject in need thereof.

The term “kidney failure” as used herein refers to is a disease that causes a progressive loss of kidney function, with a concomitant decrease in the glomerular filtration rate (GFR) or index. Kidney failure is also known as renal impairment or kidney disease. Kidney disease can be classified as (i) acute kidney injury (AKI), a progressive loss of kidney function, which generally causes oliguria and a fluid and electrolyte imbalance and (ii) chronic kidney disease (CKD), a much slower loss of kidney function over a period of months or years. Depending on the degree of kidney function, five stages of CKD are defined on the basis of the GFR: (a) stage 1, normal or high GFR (>90 ml/min), (b) stage 2: Mild CKD, GFR=60-89 mi/min, (c) stage 3, moderate CKD, GFR=30-59 m/min, (d) stage 4, severe CKD, GFR=15-29 ml/min and (e) stage 5, terminal CKD, GFR<15 ml/min. In stage 5, dialysis or a kidney transplant are required to maintain the state of health. AKI and CKD may occur concomitantly, which is known as acute-on-chronic renal failure.

In some aspects, the compounds, pharmaceutical compositions, combined preparations, methods and routes of administration of the present invention can be used to increase tissular perfusion in a subject with kidney disease. The kidney disease in the subject can be acute, chronic or both. In some aspects, the subject is undergoing dialysis (e.g., peritoneal, hemodialysis). In a further form of this aspect, the subject is undergoing hemodialysis. In some other aspects, the subject is not dialyzed (e.g., a subject with CKD in stages 1 to 4). In one version of this aspect, the subject is administered an inositol phosphate of the present invention (e.g., myo-inositol hexaphosphate) in an effective dosage of about 0.001 mg/kg to about 60 mg/kg.

In some aspects, the compounds, pharmaceutical compositions, combined preparations, methods and routes of administration of the present invention can be used for treating or preventing ischemia and/or an ischemia-related disease or condition in a subject with kidney disease. The kidney disease in the subject can be acute, chronic or both. In one version of this aspect, the subject is undergoing dialysis (e.g., peritoneal, hemodialysis). In a further form of this version, the subject is undergoing hemodialysis. In another version of this aspect, the subject is not dialyzed (e.g., a subject with CKD in stages 1 to 4). In one version of this aspect, the subject is administered an inositol phosphate of the present invention (e.g., myo-inositol hexaphosphate) in an effective dosage of about 0.001 mg/kg to about 60 mg/kg.

In some aspects, the compounds, pharmaceutical compositions, combined preparations, methods and routes of administration of the present invention can be used for improving the walking ability of a subject in need thereof. In some aspects, the compounds, pharmaceutical compositions, methods and routes of administration of the present invention can be used for increasing the Maximal Walking Distance (MWD), Maximal Walking Time (MWT) or both in a subject in need thereof. In some aspects, the subject is affected with kidney disease. The kidney disease in the subject can be acute, chronic or both. In one version of this aspect, the subject is undergoing dialysis (e.g., peritoneal, hemodialysis). In a further form of this version, the subject is undergoing hemodialysis. In another version of this aspect, the subject is not dialyzed (e.g., a subject with CKD in stages 1 to 4). In one version of this aspect, the subject is administered an inositol phosphate of the present invention (e.g., myo-inositol hexaphosphate) in an effective dosage of about 0.001 mg/kg to about 60 mg/kg.

The compounds, pharmaceutical compositions, combined preparations, methods and routes of administration of the present invention are particularly useful for increasing tissular perfusion and/or oxygenation in the lower limbs and, especially, for the treatment and prevention of peripheral artery disease. A further condition that can benefit from the use of the inositol phosphates of the present invention is critical limb ischemia. In particular aspects the compounds, pharmaceutical compositions, combined preparations, methods and routes of administration as defined in any of the aspects disclosed above are for use in increasing tissular perfusion and/or oxygenation, especially, in the lower limbs and, especially, for the treatment and prevention of PAD and/or CLI. In some aspects, the subject is undergoing dialysis (e.g., peritoneal, hemodialysis). In some further aspects, the subject is undergoing hemodialysis. In some other aspects, the subject is not dialyzed (e.g., a subject with CKD in stages 1 to 4). In one version of this aspect, the subject is administered an inositol phosphate of the present invention (e.g., myo-inositol hexaphosphate) in an effective dosage of about 0.001 mg/kg to about 60 mg/kg.

The following embodiments further illustrate the scope of the invention:

Embodiment 1. A compound of general formula I, or a pharmaceutically acceptable salt thereof, for use in increasing tissular perfusion and/or oxygenation in a subject in need thereof

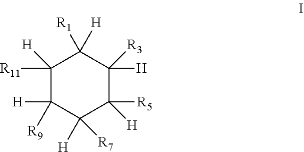

where R₁, R₃, R₅, R₇, R₉ and R₁₁ are independently selected from OH, a radical of formula II, III, IV, V, VI and VII:

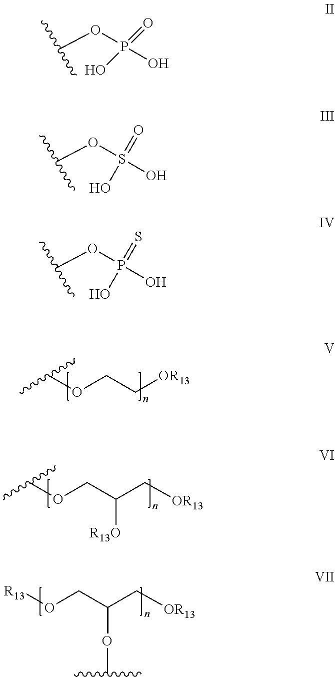

wherein: n is an integer in the range from 2 to 200, and R₁₃ is selected from H, methyl, ethyl and C₃-C₁₀ alkyl;
with the condition that:
at least one of R₁, R₃, R₅, R₇, R₉ and R₁₁ is selected from a radical of formula II, III and IV, and
zero, one, two or three of R₁, R₃, R₅, R₇, R₉ and R₁₁ is selected from a radical of formula V, VI and VII.

Embodiment 2. The compound for use according to embodiment 1, that is for the treatment or prevention of peripheral arterial disease.

Embodiment 3. The compound for use according to any of the preceding embodiments, that is for the treatment or prevention critical limb ischemia.

Embodiment 4. The compound for use according to any of the preceding embodiments, that is for treating a subject that is being subjected to dialysis, preferably hemodialysis.

Embodiment 5. The compound for use according to any of the preceding embodiments, that is a sodium salt.

Embodiment 6: The compound for use according to any of the preceding embodiments, wherein at least two, at least three, at least four, at least five or at least 6 of R₁, R₃, R₅, R₇, R₉ and R₁₁ are a selected from the radicals of formulas V, VI and VII.

Embodiment 7. The compound for use according to the preceding embodiment, wherein at least two, at least three, at least four, at least five or at least six of R₁, R₃, R₅, R₇, R₉ and R₁₁ are a radical of formula V.

Embodiment 8. The compound for use according to the preceding embodiment, wherein the compound of formula I is inositol hexaphosphate.

Embodiment 9. The compound for use according to the preceding embodiment, that is a hexasodium salt.

Embodiment 10. The compound for use according to embodiment 7, wherein:

R₇ is OSO₃⁻, and R₁, R₃, R₅, R₉ and R₁₁ are independently selected from OPO₃²⁻, OPSO₂²⁻ or OSO₃⁻.
R₉, R₅ and R₁ are OPO₃²⁻ and R₇, R₃ and R₁ are OSO₃⁻;
R₉, R₅ and R₁ are OSO₃⁻ and R₇, R₃ and R₁ are OPO₃²⁻;
R₃, R₁ and R₁ are OSO₃⁻ and R₉, R₇ and R₅ are OPO₃²⁻;
R₃, R₁ and R₁ are OPO₃²⁻ and R₉, R₇ and R₅ are OSO₃⁻;
R₇ and R₁ are OPO₃²⁻ and R₉, R₅, R₃, and R₁ are OPO₃⁻;
R₇ and R₁ are OSO₃⁻ and R₉, R₅, R₃, and R₁ are OPO₃²⁻.
R₇ and R₅ are OPO₃²⁻ and R₉, R₃, R₁, and R₁ are OSO₃⁻; or,
R₇ and R₅ are OSO₃⁻ and R₉, R₃, R₁, and R₁ are OPO₃²⁻

Embodiment 11. The compound for use according to any of the preceding embodiments, wherein the compound of formula I has myo-inositol conformation.

Embodiment 12. The compound for use according to any one of embodiments 1-6, wherein one, two or three of R₁, R₃, R₅, R₇, R₉ and R₁₁ is selected from a radical of formula V, VI and VII.

Embodiment 13. The compound for use according to the preceding embodiment, wherein four of R₁, R₃, R₅, R₇, R₉ and R₁₁ are a radical of formula II and two of R₁, R₃, R₅, R₇, R₉ and R₁₁ are selected from a radical of formulas V, VI and VII.

Embodiment 14. The compound for use according to the preceding embodiment, wherein four of R₁, R₃, R₅, R₇, R₉ and R₁₁ are a radical of formula II and two of R₁, R₃, R₅, R₇, R₉ and R₁₁ are a radical of formula V.

Embodiment 15. The compound for use according to any one of embodiments 12-14, wherein:

(i) R₁, R₅, R₉ and R₁₁ are a radical of formula II and R₃ and R₇ are selected from a radical of formulas V, VI and VII,
(ii) R₁, R₃, R₉ and R₁₁ are a radical of formula II and R₅ and R₇ are selected from a radical of formulas V, VI and VII.

Embodiment 16. The compound for use according to the preceding embodiment, wherein the radical selected from V, VI and VII is the radical of formula V.

Embodiment 17. The compound for use according to any of the embodiments 12-16, wherein the radical of formula V, VI or VI has n in the range from 2 to 200.

Embodiment 18. The compound for use according to the preceding embodiment, wherein n is the range from 9 to 30.

Embodiment 19. The compound for use according to the preceding embodiment, wherein n is the range from 15 to 30.

Embodiment 20. The compound for use according to embodiment 17, wherein n is the range from 3 to 9.

Embodiment 21. The compound for use according to any of the embodiments 12-20, wherein R₁₃ is H.

Embodiment 22. The compound for use according to embodiment 21, wherein R₁, R₅, R₉ and R₁₁ are a radical of formula II and R₃ and R₇ are a radical of formula V.

Embodiment 23. A pharmaceutical composition for the use as defined in any of the embodiments 1-4 comprising the compound as defined in any of the preceding embodiments together with pharmaceutically acceptable excipients and carriers.

Embodiment 24. The pharmaceutical composition according to the previous embodiment, wherein the compound is present at 20 to 90% (w/w) of the total composition.

Embodiment 25. The pharmaceutical composition according to the previous embodiment, wherein the compound is present at 30 to 80% (w/w) of the total composition.

Embodiment 26. The pharmaceutical composition according to the previous embodiment, wherein the compound is present at 40 to 70% (w/w) of the total composition.

Embodiment 27. The pharmaceutical composition according to any of the embodiments 23-26, wherein the composition is in dry form for reconstitution with a suitable vehicle.

Embodiment 28. The pharmaceutical composition according to any of the embodiments 23-26, wherein the composition is in solution, preferably isotonic saline solution.

Embodiment 29. The pharmaceutical composition according to any of the embodiments 23-27, that forms part of a hemodialysis, hemofiltration, or peritoneal dialysis solution.

Embodiment 30. The pharmaceutical composition according to any of the embodiments 23-29, wherein the composition is for controlled release.

Embodiment 31. The compound for use according to any one of the embodiments 1-22 or the pharmaceutical composition for use according to any of the embodiments 23-30, that is administered to a patient that is being subjected to dialysis.

Embodiment 32. The compound for use according to the previous embodiment, wherein the dialysis is hemodialysis.

Embodiment 33. The compound or pharmaceutical composition for use according to any of the embodiments 31-32, that is administered before the dialysis.

Embodiment 34. The compound or pharmaceutical composition for use according to any of the embodiments 31-32, that is administered during the dialysis.

Embodiment 35. The compound or pharmaceutical composition for use according to any of the embodiments 31-32, that is administered after the dialysis.

Embodiment 36. The compound or pharmaceutical composition for use according to any of the embodiments 31-35, that is administered by parenteral route.

Embodiment 37. The compound or pharmaceutical composition for use according to the preceding embodiment wherein the parenteral administration is intravenous, subcutaneous or intramuscular.

Embodiment 38. The compound or pharmaceutical composition for use according to the preceding embodiment, wherein the intravenous administration is by bolus injection or by intravenous infusion.

Embodiment 39. The compound or pharmaceutical composition for use according to embodiment 34, that is administered to the unfiltered blood extracted from the patient.

Embodiment 40. The compound or pharmaceutical composition for use according to any of the preceding embodiments, wherein the compound is administered to the subject in a therapeutically effective dosage of about 0.001 mg/kg to about 60 mg/kg.

Embodiment 41. The compound or pharmaceutical composition for use according to embodiment 40, wherein the compound is administered to the subject in a therapeutically effective dosage of about 15 mg/kg to about 45 mg/kg.

Embodiment 42. A combined preparation comprising: (i) (a) at least one compound according to any one of the embodiments 1-22 or (b) at least one pharmaceutical composition according to any of the embodiments 23-30, and (ii) at least one additional therapeutic agent for use in human health.

Embodiment 43. The combined preparation according to the preceding embodiment wherein the additional therapeutic agent is cilostazol, pentoxifylline or a combination thereof.

Embodiment 44. A method for increasing tissular perfusion and/or oxygenation in a subject in need thereof which comprises administering a therapeutically effective amount of the compound or pharmaceutical composition according to any one of embodiments 1-41 or the combined preparation according to any one of the embodiments 42-43 to the subject.

Embodiment 45. A method for treating or preventing ischemia and/or an ischemia-related disease or condition in a subject in need thereof which comprises administering a therapeutically effective amount of the compound or pharmaceutical composition according to any one of embodiments 1-41 or the combined preparation according to any one of the embodiments 42-43 to the subject.

Embodiment 46. A method for improving the walking ability in a subject in need thereof which comprises administering a therapeutically effective amount of the compound or pharmaceutical composition according to any one of embodiments 1-41 or the combined preparation according to any one of the embodiments 42-43 to the subject.

Embodiment 47. A method for increasing the Maximal Walking Distance (MWD), Maximal Walking Time (MWT) or both in a subject in need thereof which comprises administering a therapeutically effective amount of the compound or pharmaceutical composition according to any one of embodiments 1-41 or the combined preparation according to any one of the embodiments 42-43 to the subject.

Embodiment 48. A method for treating or preventing peripheral arterial disease in a subject in need thereof which comprises administering a therapeutically effective amount of the compound or pharmaceutical composition according to any one of embodiments 1-41 or the combined preparation according to any one of the embodiments 42-43 to the subject.

Embodiment 49. The methods according to any one of embodiments 44-48 wherein the combined preparation is administered jointly, concurrently or sequentially to the subject.

This invention is further illustrated by the following examples which should not be construed as limiting. The contents of all documents cited throughout this application are incorporated herein in their entirety by reference.

### General Procedures

1. Limb Blood Perfusion

Position limb blood perfusion and ischemia status (i.e., blood perfusion including, perfusion unit, perfusion difference and perfusion ratio) is evaluated by laser doppler perfusion imaging using a PeriCam PSI NR imager (Perimed AB, Jarfalla, SE). Subjects are anesthetized using 3% isoflurane delivered in 100% oxygen at a flow rate at 1 L/min before measuring. The perfusion difference and perfusion ratio are calculated by comparing the baseline and any indicated interim or final readings for each group. Blood flux is assessed around the Cₘₐₓ of the tested active agents (i.e., 15 min after treatment with SNF472, 20 min after treatment with IP4-BIS-PEG100, and 3 to 4 hours after treatment with cilostazol).

2. Walking Ability Tests

Quantification of walking ability (maximal waling time (MWT) and maximal walking distance (MWD)) are measured by a forced incremental treadmill running test. A two-lane rodent treadmill (LE8709TS; PanLab/Harvard Apparatus, Holliston, Mass., US) is employed. The treadmill is maintained at a 15% inclination running at a speed of 15 m/min (25 cm/sec) during the first 5 min, then at 33 cm/sec during the following 5 min, and finally, at a speed of 40 cm/sec for a maximum of 30 additional minutes.

Limb function is assessed around the Cₘₐₓ of the tested active agents (i.e., 15 min after treatment with SNF472, and 3 to 4 hours after treatment with cilostazol). Subjects are exercised in the treadmill for acclimatization according to a set protocol prior to testing. Subjects that do not comply with the protocol are excluded from the test.

Subjects are kept running for up to 40 min or until exhausted (i.e., they remain on the shock grid for five continuous seconds) during the test. MWD and MWT are then calculated for each animal.

3. Tissue and Blood Collection, Calcification Assays

Subjects are anesthetized by isoflurane inhalation. Blood is obtained by exsanguination through cardiac puncture. The subjects are then sacrificed and their tissues (e.g., right and left femoral arteries, aorta) are collected. The blood and tissues are processed and analyzed for determining their calcium contents.

Calcium content in tissue sample is quantified via inductively coupled plasma optical emission spectrometry (ICP-OES) using an Optima 7300 DV ICP-OES System spectrometer (PerkinElmer, Inc., Waltham, Mass., US) according to the manufacturer's instructions. Myo-inositol-hexaphosphate levels in plasma are quantified by LC-MS/MS chromatographic methods described in the art. See WO2013050603.

### Example 1

### Prevention of Limb Ischemia

### Impact in Blood Perfusion

The preventive effects of SNF472, IP4-4,6-bisPEG100 sodium salt and cilostazol on blood perfusion were tested using a rat model for a duration of 12 days. Limb ischemia in the subjects was induced from D1 excepting the sham group, to which no ischemia was induced. Subjects induced with ischemia were then treated with placebo and active agent formulations from D1 to D12 to assess their impact in preventing limb ischemia. Observations were taken at several points during treatment from D1 to D12. All subjects were weighed every day before treatment.

1. Induction of Limb Ischemia

Fifty-four male Sprague Dawley (SD) rats (Envigo Corp., Huntingdon, GB) weighing approximately 250-275 g were used. The subjects were fed with an A04 diet (Scientific Animal Food & Engineering; Carpe Bio, Amersfoort, NL). The subjects were divided in 5 groups, 8 to 10 rats per group, as follows:

Group 1—Control (sham)

Group 2a Placebo—Physiological saline solution

Group 2b Placebo—5% carboxymethyl cellulose (CMC) sodium salt solution

Group 3—SNF472 (Na₆IP₆)

Group 4—IP4-4,6-bisPEG100 sodium salt

Group 5—Cilostazol (C₂₀H₂₇N₅O₂)

Limb ischemia was induced in the subjects of groups 2a-5 by subcutaneous administration of 120,000 IU/kg vitamin D3 (cholecalciferol, Duphafral D₃ 1000; Zoetis Inc., Parsippany, N.J., US) in physiological saline solution (2 mL/kg) every day during D1 to D3. The sham group 1 subjects were administered physiological saline solution (2 mL/kg) subcutaneously every day during D1 to D3 with no vitamin D3.

The subjects in groups 1 and 2a were administered physiological saline solution (2 mL/kg) subcutaneously every day during D1 to D12. The subjects in group 2b were administered a 5% CMC sodium salt in water solution (5 mL/kg) orally every day during D1 to D12.

The administration of vitamin D3 induced ischemia in the posterior limbs of the subjects in groups 2a, 2b, and 3-5 was assayed by laser doppler perfusion imaging.

2. Ischemia Rescue and Effects on Limb Blood Perfusion—Laser Doppler Imaging Assay

Limb blood perfusion was induced in the subjects of groups 3 and 4 by subcutaneous administration of 20 mg/kg of SNF472 (Na₆IP₆; free base: 600 g/mol) and IP4-4,6-bisPEG100 sodium salt (free base 696.27 g/mol), respectively, in physiological saline solution (2 mL/kg) every day during D1 to D12. Limb blood perfusion was induced in the group 5 subjects by oral administration of 20 mg/kg cilostazol (C₂₀H₂₇N₅O₂, free base 369.46 g/mol; lot no. LRAB9590, Sigma-Aldrich Corp., St. Louis, Mo., US) in a 5% CMC sodium salt in water solution (5 mL/kg) every day during D1 to D12.

Limb ischemia status was evaluated at days D0 (baseline), D6 and D12 in all rats by laser doppler perfusion imaging. The perfusion difference and perfusion ratio were calculated by comparing the D0 baseline and either the D6 and D12 readings for each group. In particular, the perfusion difference and perfusion ratio were calculated by comparing the group 1 (control) and (a) groups 2a and 2b placebo (i.e., physiological saline solution, 5% CMC sodium salt in water solution), (b) group 3 (SNF472), (c) group 4 (IP4-4,6-bisPEG100 sodium salt), and (d) group 5 (cilostazol) readings.

On the days when blood perfusion tests involving the use of active agents were conducted (i.e., D6, D12), the dosages were administered as follows:

Group 3—SNF472 (Na₆IP₆): 15 minutes before reading

Group 4—IP4-4,6-bisPEG100 sodium salt: 20 minutes before reading

Group 5—Cilostazol (C₂₀H₂₇N₅O₂): 3 to 4 hours before reading

Under the scheme above, the active agents would be at their maximum serum concentration (Cₘₐₓ) at the time when the test readings were taken.

The group 1 subjects did not show any significant changes in their perfusion parameters.

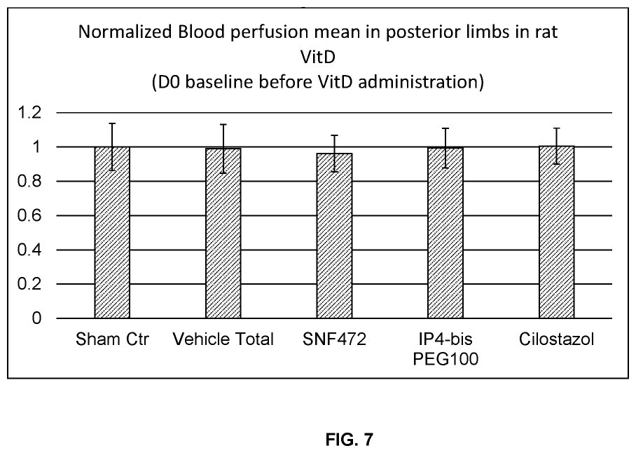

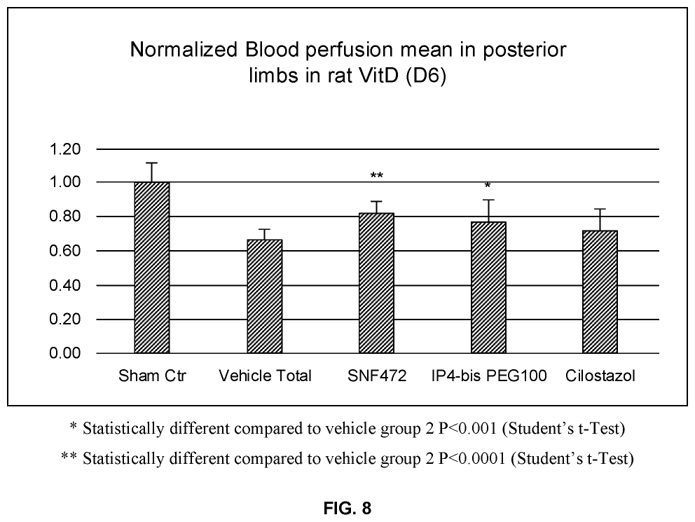

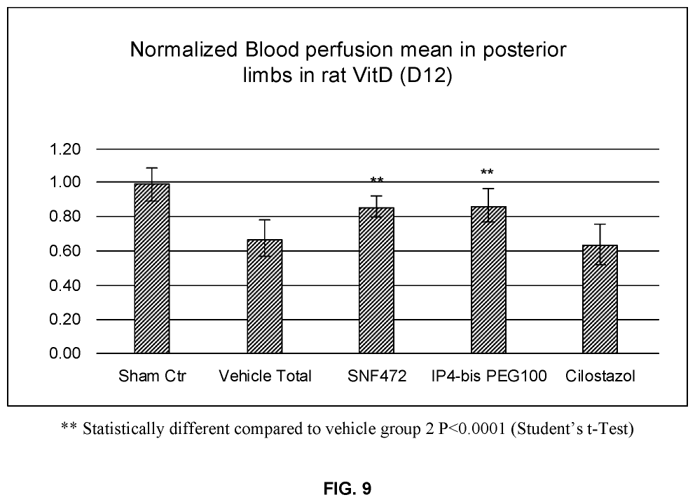

The administration of SNF472 and the IP4-4,6-bisPEG100 sodium salt attenuated the drop in blood perfusion in the limbs of the subjects of groups 3-5 (i.e., treated with vitamin D3) compared to both placebo and cilostazol. These results suggest that SNF472 and IP4-4,6-bisPEG100 are more effective than cilostazol for increasing blood perfusion in the posterior limbs of the treated subjects. See FIGS. 7, 8, and 9.

3. Calcium Content and Calcification—ICP-OES

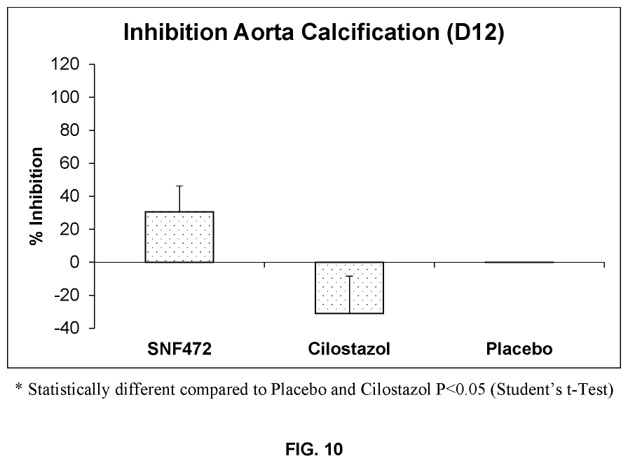

SNF472 showed to be effective against vascular calcification, as SNF472 inhibited aorta calcification (31±16%) after a daily subcutaneous dosing of 20 mg/kg compared to placebo. Cilostazol was not active against calcification at the same dose. See FIG. 10.

4. Pharmacokinetics

The subjects from groups 1, 2, 3 and 5 were sacrificed after exsanguination. Then, their necropsies were performed, and their aortas were collected. The tissues were lyophilized for 24 h and weighed. The lyophilized tissues were then digested using a 1:1 HNO₃:HClO₄ mixture in a dry bath incubator for 2-4 h at 180° C. The digested tissues were subsequently diluted using ultra-pure Milli-Q water (MilliporeSigma (Merck KGaA), Burlington, Mass., US) to a final volume of 10 mL. The calcium content in the aorta sample was quantified via ICP-OES.

Subjects from groups 1, 2, 3, 4 and 5 were anesthetized and their blood was obtained in D12. Around 8-10 mL of total blood per subject were taken and divided in collection tubes for plasma (K3EDTA, approximately 6 mL blood) and serum (approximately 2-3 mL blood) testing. Plasma was stored in one aliquot of 600 μL and several other aliquots of 500 μL. Serum was divided in two aliquots.

Myo-inositol-hexaphosphate levels in the plasma of the subjects of group 3 (i.e., taken at around Cₘₐₓ, 15 min after the last SNF472 dosing) were quantified by LC-MS/MS chromatographic methods described in the art. See WO2013050603.

All subjects in group 3 were well exposed to the SNF472 product. The plasma level 15 min after the last subcutaneous dosing in D12 was 15587±ng/mL (24±12 μM). The plasma levels in rats after a daily dosing at 20 mg/kg were comparable to the levels found in hemodialysis patients treated with SNF472 at a dose of 4.2 mg/kg via the intravenous route.

### Example 2

### Prevention of Limb Ischemia

### Impact on Maximal Walking (MWT) and Distance (MD T) Times

The preventive effects of SNF472 and cilostazol on blood perfusion, walking ability and tissue calcification were tested using a rat model for a duration of 24 days. Additionally, the effects of a combined treatment of SNF472 and cilostazol on blood perfusion were also tested. Limb ischemia in the subjects was induced from D1 to D3 excepting the sham group, to which no ischemia was induced. Subjects induced with ischemia were then treated with placebo and active agent formulations from D1 to D12 to assess their impact in preventing (a) limb ischemia and tissue calcification and (b) the deterioration in walking ability. Treatment in all subjects was discontinued from D13 to D24. Observations were taken at several points during the treatment and post-treatment phases from D1 to D24. All subjects were weighed every day before treatment.

1. Induction of Limb Ischemia

Fifty-four male Sprague Dawley (SD) rats (Envigo Corp., Huntingdon, GB) weighing approximately 250-275 g were used. The subjects were fed with an A04 diet (Scientific Animal Food & Engineering; Carpe Bio, Amersfoort, NL). The subjects were divided in 5 groups, 8 to 10 animals per group, as follows:

Group 1—Control (sham)

Group 2a Placebo—Physiological saline solution

Group 2b Placebo—5% CMC sodium salt solution

Group 3—SNF472 (Na₆IP₆)

Group 4—Cilostazol (C₂₀H₂₇N₅O₂)

Group 5—Cilostazol+SNF472

Limb ischemia was induced in the subjects of groups 2a-5 by subcutaneous administration of 120,000 IU/kg vitamin D3 (cholecalciferol, Duphafral D₃ 1000; Zoetis Inc., Parsippany, N.J., US) in physiological saline solution (2 mL/kg) every day during D1 to D3. The sham group 1 subjects were administered physiological saline solution (2 mL/kg) subcutaneously every day from D1 to D3 with no vitamin D3.

The subjects in groups 1 and 2a were administered physiological saline solution (2 mL/kg) subcutaneously every day during D1 to D12. The subjects in group 2b were administered a 5% CMC sodium salt in water solution (5 mL/kg) orally every day during D1 to D12.

2. Ischemia Rescue and Effects on Blood Perfusion—Laser Doppler Imaging Assay

Subjects in groups 1 and 2a were administered 2 mL/kg of physiological saline solution daily from D1 to D12 via subcutaneous route. Subjects in group 2b were administered orally 5 mL/kg of a 5% CMC sodium salt in water solution daily from D1 to D12. In addition, subjects in group 3 were administered 20 mg/kg of SNF472 (Na₆IP₆, free base: 600 g/mol) sodium salt (free base 696.27 g/mol) in physiological saline solution (2 mL/kg) every day from D1 to D12 via subcutaneous route. Subjects in group 4 were administered orally 20 mg/kg cilostazol (C₂₀H₂₇N₅O₂, free base 369.46 g/mol; lot no. LRAB9590, Sigma-Aldrich Corp., St. Louis, Mo., US) in a 5% CMC sodium salt in water solution (5 mL/kg) daily from D1 to D12. Subjects in group 5 were administered (i) 20 mg/kg cilostazol (C₂₀H₂₇N₅O₂, free base 369.46 g/mol; lot no. LRAB9590, Sigma-Aldrich Corp., St. Louis, Mo., US) in a 5% CMC sodium salt in water solution (5 mL/kg) orally, followed by (ii) 20 mg/kg of SNF472 (Na₆IP₆, free base: 600 g/mol) in physiological saline solution (2 mL/kg) via subcutaneous route. The administrations were performed daily from D1 to D12.

On the days when blood perfusion tests involving the use of active agents were conducted (i.e., D6, D12, and D18), the dosages were administered as follows:

Group 3—SNF472 (Na₆IP₆): 15 minutes before reading

Group 4—Cilostazol (C₂₀H₂₇N₅O₂): 3 to 4 hours before reading

Group 5—Cilostazol+SNF472: 15 minutes for SNF472 and 3 to 4 hours for cilostazol before reading

Under the scheme above the active agents would be at their maximum plasma concentration (Cₘₐₓ) at the time when the test readings were taken.

Limb ischemia status was evaluated during treatment and after treatment was interrupted (i.e., D0, D6, D12, D18) in all rats by laser doppler perfusion imaging. The perfusion difference and perfusion ratio were calculated by comparing the baseline and either of the D6, D12, and D18 readings for each group. In particular, the perfusion difference and perfusion ratio were calculated by comparing the group 1 (control) and (a) groups 2a and 2b placebo (physiological saline solution, 5% CMC sodium salt solution), (b) group 3 (SNF472), (c) group 4 (cilostazol), and (d) group 5 (cilostazol/SNF472 combination) readings.

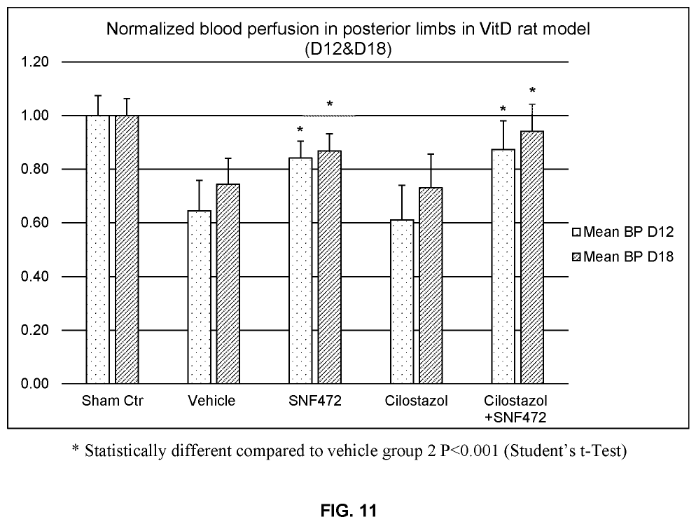

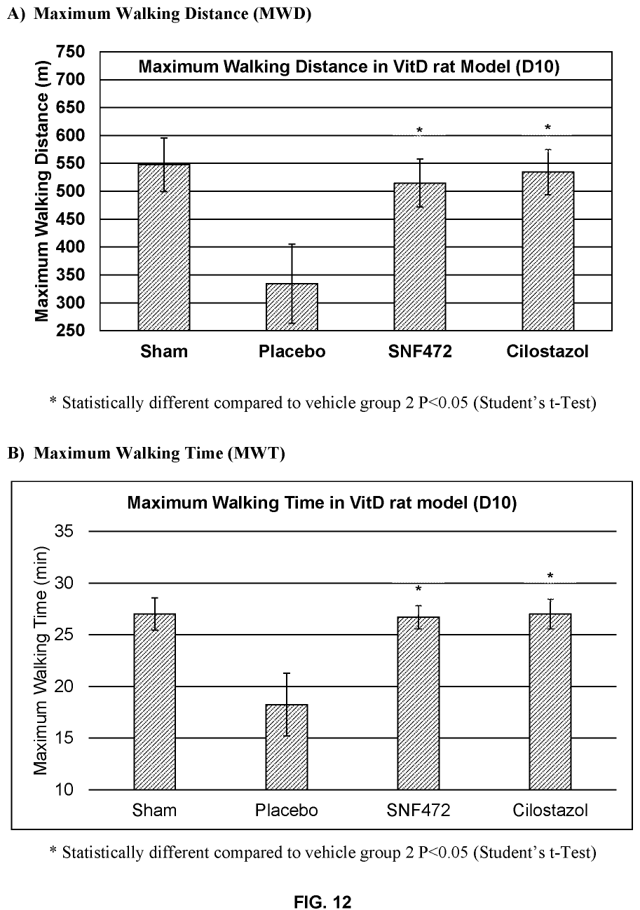

SNF472 by itself or in combination with cilostazol attenuated rat limb ischemia. Treatment with cilostazol alone was not effective in this model. The SNF472 effects on blood perfusion were maintained even 6 days after interrupting treatment. See FIG. 11.

3. Effects on Walking Ability—Treadmill Running Test

Maximal waling time (MWT) and maximal walking distance (MWD) were assessed in groups 1-4 by the treadmill running test (8 to 10 animals per group and time point) in D0, D5, D10, and D17.

Subjects were acclimatized to the treadmill for two days before conducting the test. On the first day, subjects were exercised for 5 to 10 minutes with the treadmill speed ranging progressively from 15 m/min to 24 m/min. On the second day, the subjects were exercised initially at a speed of 15 m/min for the 5 minutes. Then, they were exercised at 19.8 m/min (33 cm/sec) for 5 additional minutes. Finally, the subjects were exercised at 24 m/min (40 cm/sec) for a maximum 30 additional minutes. Preoperative walking time and distance recordings were obtained. Animals that did not comply with the protocol were excluded from the test.

Limb function (MWT and MWD) was assessed in groups 1, 2, and 3 using the treadmill running test 15 min after the corresponding daily administration. Limb function in groups 4 was assessed from 3 to 4 hours after treatment.

Under the scheme above the active agents would be at their maximum plasma concentration (Cₘₐₓ) at the time when the running test readings were taken.

The subjects were kept running for 40 minutes or until exhausted (i.e., they remain on the shock grid for five continuous seconds). MWD and MWT was then calculated for each animal.

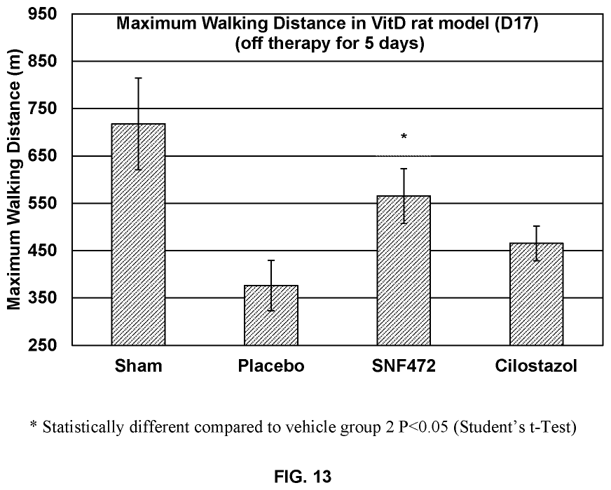

SNF472 and cilostazol improved walking ability in rats compared to vehicle (+54% MWD and +46% MWT). See FIG. 13. Moreover, the effect of SNF472 on improving walking ability was maintained even 5 days after interrupting treatment (D17). Contrarily, cilostazol lost its beneficial effects immediately after treatment was discontinued. See FIG. 13.

4. Calcium Content and Calcification—ICP-OES

Subjects were anesthetized and their blood was obtained in D24. The subjects were sacrificed after exsanguination. Then, their necropsies were performed, and their aortas were collected. The tissues were lyophilized for 24 h and weighed. The lyophilized tissues were then digested using a 1:1 HNO₃:HClO₄ mixture in a dry bath incubator for 2-4 h at 180° C. The digested tissues were subsequently diluted using ultra-pure Milli-Q water (MilliporeSigma (Merck KGaA), Burlington, Mass., US) to a final volume of 10 mL. The calcium content in the tissue samples was quantified via ICP-OES.

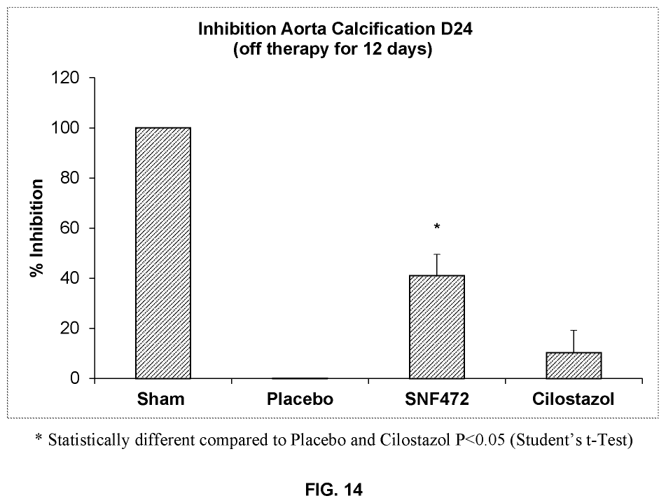

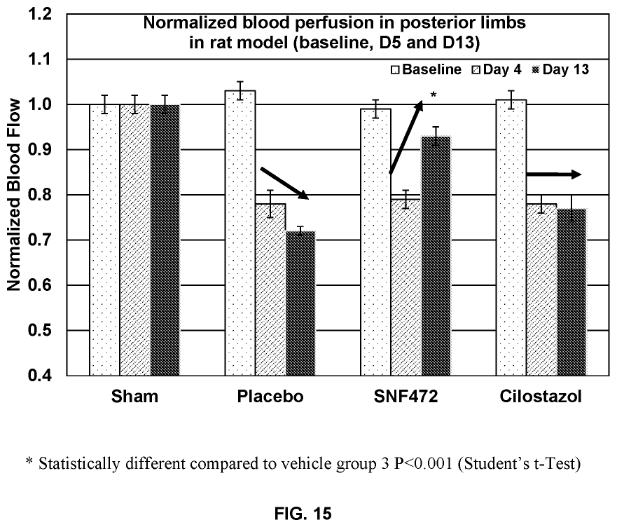

SNF472 showed to be effective against vascular calcification, as SNF472 inhibited aorta calcification (41±9%) after daily subcutaneous dosing at 20 mg/kg compared to placebo. Cilostazol was not active against calcification at the same dose. See FIG. 14.

### Example 3

### Treatment of Limb Ischemia

The effects of SNF472 and cilostazol over blood perfusion, walking ability and tissue calcification after the inception of limb ischemia were tested using a rat model for a duration of 13 days. Limb ischemia was induced to all groups from D1 to D3 excepting the sham group, to which no ischemia was induced. Subjects induced with ischemia were administered placebo and active agent formulations from D5 on to allow for the development of ischemia before starting treatment. From D5 to D13, subjects were treated from for assessing the impact of treatment on limb ischemia, walking ability and tissue calcification. Observations were taken at several points during the treatment from D1 to D13. All subjects were weighed every day before treatment.

1. Induction of Limb Ischemia

Sixty-six male Sprague Dawley (SD) rats (Envigo Corp., Huntingdon, GB) weighing approximately 250-275 g were used. The subjects were fed with an A04 diet (Scientific Animal Food & Engineering; Carpe Bio, Amersfoort, NL). The subjects were divided in 5 groups, 8 to 14 animals per group, as follows:

Group 1—Control (Sham)

Group 2—D5 Ca baseline

Group 3a Placebo—Physiological saline solution

Group 3b Placebo—5% CMC sodium salt solution

Group 4—SNF472 (Na₆IP₆)

Group 5—Cilostazol (C₂₀H₂₇N₅O₂)

Limb ischemia was induced in the subjects of groups 2-5 by subcutaneous administration of 120,000 IU/kg vitamin D3 (cholecalciferol, Duphafral D₃ 1000; Zoetis Inc., Parsippany, N.J., US) in physiological saline solution (2 mL/kg) every day during D1 to D3. The group 1 subjects were administered physiological saline solution (2 mL/kg) subcutaneously every day from D1 to D3 with no vitamin D3.

2. Ischemia Rescue and Effects on Blood Perfusion—Laser Doppler Imaging Assay

Subjects in group 1 were administered 2 mL/kg of physiological saline solution daily from D1 to D13 via subcutaneous route. Subjects in group 2 were administered 2 mL/kg of physiological saline solution daily from D1 to D5 via subcutaneous route. On D5, four subjects of group 1 and all the subjects of group 2 were sacrificed for determining their Ca baseline values.

Subjects in groups 3a placebo were administered 2 mL/kg of physiological saline solution daily from D5 to D13 via subcutaneous route. Subjects in group 3b placebo were administered orally 5 mL/kg of a 5% CMC sodium salt in water solution daily from D5 to D13. In addition, subjects in groups 4 were administered 40 mg/kg of SNF472 (Na₆IP₆, free base: 600 g/mol) in physiological saline solution (2 mL/kg), every day from D5 to D13 via subcutaneous route.

Subjects in group 5 were administered orally 40 mg/kg cilostazol (C₂₀H₂₇N₅O₂, free base 369.46 g/mol; lot no. LRAB9590, Sigma-Aldrich Corp., St. Louis, Mo., US) in a 5% CMC sodium salt in water solution (5 mL/kg) daily from D5 to D13. A 40 mg/kg dose of cilostazol in rats is comparable to a therapeutic dose of 8.4 mg/kg in PAD patients.

On the days when blood perfusion tests involving the use of active agents were conducted (i.e., D5, D13), the dosages were administered as follows:

Group 4—SNF472 (Na₆IP₆): 15 minutes before reading

Group 5—Cilostazol (C₂₀H₂₇N₅O₂): 3 to 4 hours before reading

Under the scheme above the active agents would be at their maximum plasma concentration (Cₘₐₓ) at the time when the test readings were taken.

Limb functional and ischemia status were evaluated by laser doppler perfusion imaging in all groups at D0 and D5, and in groups 1, 3a, 3b, 4 and 5 at D13. The perfusion difference and perfusion ratio were calculated by comparing the baseline and either D5 and D13 readings for each group. In particular, the perfusion difference and perfusion ratio were calculated by comparing the group 1 (control) and: (a) the groups 3a and 3b placebo (physiological saline solution, 5% CMC sodium salt solution), (b) the group 4 (SNF472), and (c) the group 5 (cilostazol) readings.

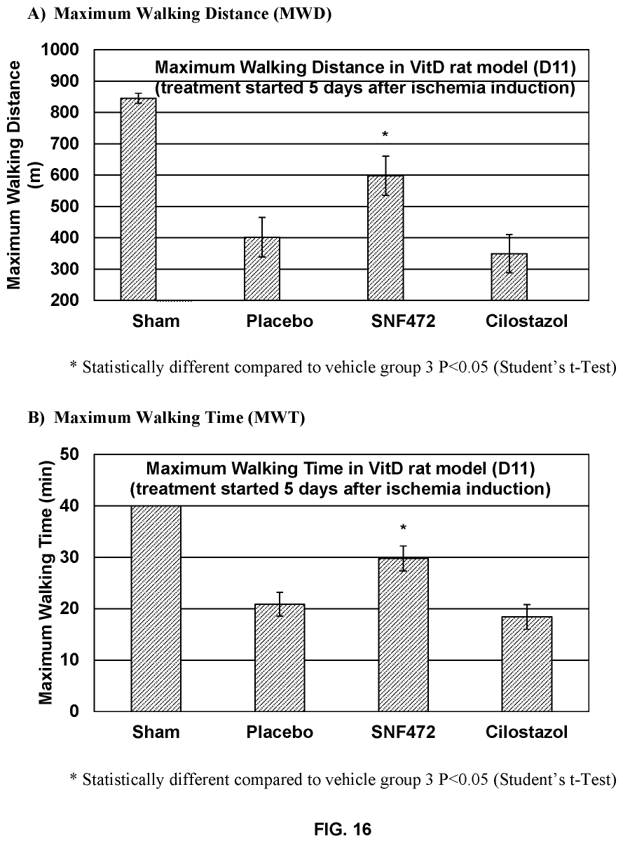

VitD3 administration induced a drop in blood perfusion in the posterior limbs in groups 3a, 3b, 4, and 5 (D5 measurement just before therapy administration). In D13, only animals treated with SNF472 showed a significant improvement of limbs blood perfusion compared to D5 before treatment. No improvement or ischemia rescue were reported in animals treated with placebo or cilostazol in D13 compared to D5. See FIG. 16.

3. Effects on Walking Ability—Treadmill Running Test

Maximal walking time (MWT) and maximal walking distance (MWD) were assessed in all groups at D0 and in groups 1, 3a, 3b, 4, and 5 at D6 and D11 by the treadmill running test (8 to 12 animals per group and time point). Subjects were acclimatized to the treadmill for two days before conducting the test. On the first day, subjects were exercised for 5 to 10 minutes with the treadmill speed ranging progressively from 15 m/min to 24 m/min. On the second day, the subjects were exercised initially at a speed of 15 m/min for the 5 minutes. Then, they were exercised at 19.8 m/min (33 cm/sec) for 5 additional minutes. Finally, the subjects were exercised at 24 m/min (40 cm/sec) for a maximum 30 additional minutes. Preoperative walking time and distance recordings were obtained. Animals that did not comply with the protocol were excluded from the test.

Limb function (MWT and MWD) was assessed in groups 1, 3a, 3b, and 4 using the treadmill running test 15 min after the corresponding daily administration. Limb function in groups 5 was assessed from 3 to 4 hours after treatment.

Under the scheme above the active agents would be at their maximum plasma concentration (Cₘₐₓ) at the time when the test readings were taken.

The subjects were kept running for 40 minutes or until exhausted (i.e., they remain on the shock grid for five continuous seconds). MWD and MWT was then calculated for each animal.

SNF472 improved rat walking ability compared to vehicle (+49% MWD) even the treatment started 5 days after ischemia induction whereas cilostazol is not effective under the same conditions and at the therapeutic dose (40 mg/kg/day). See FIG. 16.

4. Calcium Content and Calcification—ICP-OES

Four subjects of group 1 and all the subjects of group 2 were sacrificed on D5 for determining their Ca baseline values. The remaining subjects of group 1 and all the subjects of groups 3a, 3b, 4, 5, and 6 were sacrificed in D13.

The subjects were sacrificed after exsanguination. Then, their necropsies were performed, and their right and left femoral arteries were collected. The tissues were lyophilized for 24 h and weighed. The lyophilized tissues were then digested using a 1:1 HNO₃:HClO₄ mixture in a dry bath incubator for 2-4 h at 180° C. The digested tissues were subsequently diluted using ultra-pure Milli-Q water (MilliporeSigma (Merck KGaA), Burlington, Mass., US) to a final volume of 10 mL. The calcium content in the tissue samples was quantified via ICP-OES.

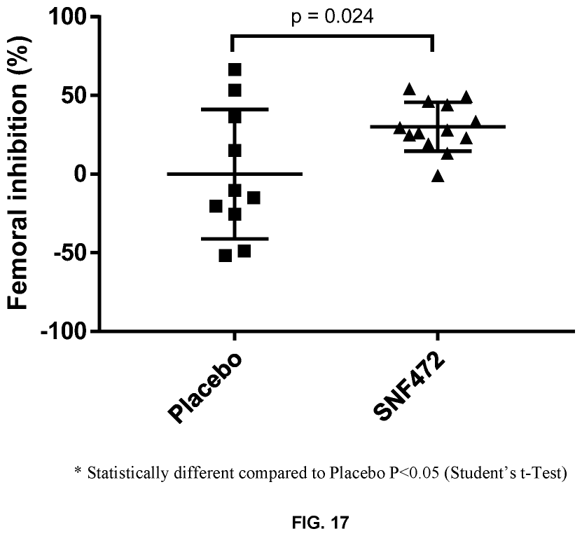

SNF472 showed to be effective against vascular calcification at D13. Compared to placebo, SNF472 inhibited calcification in femoral arteries by around (30%) after a daily subcutaneous dosing of 40 mg/kg. See FIG. 17.

5. Pharmacokinetics

Subjects from groups, 1, 3a,3b, 4, and 5 were anesthetized and their blood was obtained in D13. Around 8-10 mL of total blood per subject were taken and divided in collection tubes for plasma (K3EDTA, approximately 6 mL blood) and serum (approximately 2-3 mL blood) testing. Plasma was be stored in one aliquot of 600 μL and several other aliquots of 500 μL. Serum was be divided in two aliquots.

Myo-inositol-hexaphosphate levels in the plasma of the subjects of group 4 (i.e., taken at around Cₘₐₓ, 15 min after the last SNF472 dosing) were quantified by LC-MS/MS chromatographic methods described in the art. See WO2013050603.

All subjects in group 4 were well exposed to the SNF472 product. The plasma level 15 min after the last subcutaneous dosing in D13 was 40078±15024 ng/mL (60.7±22.8 uM). The plasma levels in rats after a daily dosing at 20 mg/kg were comparable to the levels found in hemodialysis patients treated with SNF472 at a dose of 8.4 mg/kg via the intravenous route.

### Example 4

### SN472 Drug Interactions

The compatibility of SNF472 with other drugs prescribed regularly to subjects with renal impairment was analyzed.

Wistar rats (Charles River Labs, Inc., Wilmington, Mass., US), were treated with: (i) SNF472 administered subcutaneously (s.c.), (ii) SNF472+sevelamer (oral), (iii) SNF472 (s.c.)+cinacalcet (oral), (iv) SNF472(s.c.)+Vit D (s.c.), (v) SNF472 (s.c.)+sodium thiosulfate (s.c.), and SNF472 (s.c.)+ibandronate (s.c.). No significant differences were observed between the administration of SNF472 alone or jointly with any other of the assayed drugs.

### Example 5

### Prevention of Limb Ischemia

### SNF472 Dose-Response Impact Over Maximal Walking (MWT) and Distance (MDT) Times

The preventive effects of several different doses of SNF472 and the highest tolerated cilostazol dose on blood perfusion, walking ability and tissue calcification were tested for 12 days using a rat model. Limb ischemia was induced in the subjects from D1 to D3, excepting the sham group. Animals induced with ischemia were then treated with placebo and active agent formulations from D1 to D12 to assess their impact in preventing (a) limb ischemia and tissue calcification and (b) the deterioration in walking ability. Observations were taken at several points during the treatment phase from D1 to D12. All subjects were weighed every day before treatment.

1. Induction of Limb Ischemia

One hundred and two male Sprague Dawley (SD) rats (Envigo Corp., Huntingdon, GB) weighing approximately 250-275 g were used. The subjects were fed with an A04 diet (Scientific Animal Food & Engineering; Carpe Bio, Amersfoort, NL). The subjects were divided in 5 groups, 8 to 12 animals per group, as follows:

Group 1—Control (sham)

Group 2a Placebo—Physiological saline solution, subcutaneous

Group 2b Placebo—5% CMC sodium salt solution, oral

Group 3—SNF472 (Na₆IP₆) at 1 mg/kg, subcutaneous

Group 4—SNF472 (Na₆IP₆) at 7.5 mg/kg, subcutaneous

Group 5—SNF472 (Na₆IP₆) at 15 mg/kg, subcutaneous

Group 6—SNF472 (Na₆IP₆) at 30 mg/kg, subcutaneous

Group 7—SNF472 (Na₆IP₆) at 45 mg/kg, subcutaneous

Group 8—Cilostazol (C₂₀H₂₇N₅O₂) at 45 mg/kg, oral

Limb ischemia was induced in the subjects of groups 2-8 by subcutaneous administration of 120,000 IU/kg vitamin D3 (cholecalciferol, Duphafral D₃ 1000; Zoetis Inc., Parsippany, N.J., US) in physiological saline solution (2 mL/kg) every day during D1 to D3. The sham group 1 subjects were administered physiological saline solution (2 mL/kg) subcutaneously every day from D1 to D3 with no vitamin D3.

The subjects in groups 1 and 2a were administered physiological saline solution (2 mL/kg) subcutaneously every day during D1 to D12. The subjects in group 2b were administered a 5% CMC sodium salt in water solution (5 mL/kg) orally every day during D1 to D12.

2. Ischemia Rescue and Effects on Blood Perfusion—Laser Doppler Imaging Assay

Subjects in groups 1 and 2a were administered 2 mL/kg of physiological saline solution daily from D1 to D12 via subcutaneous route. Subjects in group 2b were administered orally 5 mL/kg of a 5% CMC sodium salt in water solution every day from D1 to D12. In addition, subjects in group 3, 4, 5, 6 and 7 were administered 1 mg/kg, 7.5 mg/kg, 15 mg/kg, 30 mg/kg and 45 mg/kg of SNF472 (Na₆IP₆, free base: 600 g/mol) sodium salt (free base 696.27 g/mol), respectively. SNF472 was administrated in physiological saline solution (2 mL/kg) every day from D1 to D12 via subcutaneous route. Subjects in group 8 were administered orally 45 mg/kg cilostazol (C₂₀H₂₇N₅O₂, free base 369.46 g/mol; lot no. LRAB9590, Sigma-Aldrich Corp., St. Louis, Mo., US) in a 5% CMC sodium salt in water solution (5 mL/kg) daily from D1 to D12.

On the days when blood perfusion tests involving the use of active agents were conducted (i.e., D6 and D12), the dosages were administered as follows:

Groups 3, 4, 5, 6 and 7—SNF472 (Na₆IP₆): 15 minutes before reading

Group 8—Cilostazol (C₂₀H₂₇N₅O₂): 3 to 4 hours before reading

Under the scheme above the active agents would be at their maximum plasma concentration (Cₘₐₓ) at the time when the test readings were taken.

Limb ischemia status was evaluated during the treatment period (i.e., D0, D6 and D12) by laser doppler perfusion imaging in all rats. The perfusion difference and perfusion ratio were calculated by comparing the baseline and either of the D6 and D12 readings for each group. In particular, the perfusion difference and perfusion ratio were calculated by comparing the group 1 (control) and (a) groups 2a and 2b placebo (physiological saline solution, 5% CMC sodium salt solution), (b) groups 3, 4, 5, 6 and 7 (SNF472) and (c) group 8 (cilostazol), readings.

SNF472 attenuated rat limb ischemia with dose-response manner. Treatment with cilostazol alone was not effective in this model.

3. Effects on Walking Ability—Treadmill Running Test

Maximal walking time (MWT) and maximal walking distance (MWD) were assessed in groups 1-8 by the treadmill running test (8 to 12 animals per group and time point) in D0, D5 and D10.

Subjects were acclimatized to the treadmill for two days before conducting the test. On the first day, subjects were exercised for 5 to 10 minutes with the treadmill speed ranging progressively from 15 m/min to 24 m/min. On the second day, the subjects were exercised initially at a speed of 15 m/min for the 5 minutes. Then, they were exercised at 19.8 m/min (33 cm/sec) for 5 additional minutes. Finally, the subjects were exercised at 24 m/min (40 cm/sec) for a maximum 30 additional minutes. Preoperative walking time and distance recordings were obtained. Animals that did not comply with the protocol were excluded from the test.

Limb function (MWT and MWD) was assessed in groups 1, 2, 3, 4, 5, 6 and 7 using the treadmill running test 15 min after the corresponding daily administration. Limb function in group 8 was assessed from 3 to 4 hours after treatment.

Under the scheme above the active agents would be at their maximum plasma concentration (Cₘₐₓ) at the time when the running test readings were taken.

The subjects were kept running for 40 minutes or until exhausted (i.e., they remain on the shock grid for five continuous seconds). MWD and MWT was then calculated for each animal.

SNF472 and cilostazol improved walking ability in rats compared to vehicle. Moreover, the effect of SNF472 on improving walking ability was dose-response dependent.

4. Calcium Content and Calcification—ICP-OES

Subjects were anesthetized and their blood was obtained in D12. The subjects were sacrificed after exsanguination. Then, their necropsies were performed, and their heart and aorta arteries were collected. The tissues were lyophilized for 24 h and weighed. The lyophilized tissues were then digested using a 1:1 HNO₃:HClO₄ mixture in a dry bath incubator for 2-4 h at 180° C. The digested tissues were subsequently diluted using ultra-pure Milli-Q water (MilliporeSigma (Merck KGaA), Burlington, Mass., US) to a final volume of 10 mL. The calcium content in the tissue samples was quantified via ICP-OES.

SNF472 showed to be effective against heart and vascular calcification, such as, for example, heart and aorta arteries calcification, in a dose-response manner. Cilostazol was not active against calcification at the same dose.

### Example 6

### Prevention of Limb Ischemia in an Adenine-Induced Uremic Rat Model

### SNF472 Impact on Limb Blood Perfusion

The preventive effects of SNF472 and cilostazol doses on blood perfusion and tissue calcification were tested using a relevant chronic kidney disease rat model for a duration of 21 days.

Uremia and limb ischemia in the animals were induced from D1 to D21 excepting the sham group, to which neither uremia nor ischemia was induced. Animals induced with ischemia were then treated with placebo and active agent formulations from D1 to D21 to assess their impact in preventing (a) limb ischemia and (b) tissue calcification. Observations were taken at several points during the treatment phase from D1 to D21. All subjects were weighed every day before treatment.

1. Induction of Uremia and Limb Ischemia

Sixty-eight male Sprague Dawley (SD) rats (Envigo Corp., Huntingdon, GB) weighing approximately 250-275 g were used. fed a pelleted high-phosphorus diet (SM R, 10 mm pellets, 1.06% Ca, 1.03% P) (SSNIFF Spezialdiaeten, Soest, DE) ad libitum. The subjects were divided in 6 groups, 8 to 12 animals per group, as follows:

Group 1—Control (sham)

Group 2a Placebo—Physiological saline solution subcutaneous, once a day

Group 2b Placebo—Physiological saline solution, subcutaneous, Alzet pump 4 weeks

Group 3—SNF472 (Na₆IP₆) at 30 mg/kg, subcutaneous, once a day

Group 4—SNF472 (Na₆IP₆) at 45 mg/kg, subcutaneous, once a day

Group 5—SNF472 (Na₆IP₆) at total dose of 400 mg/4 weeks (100 mg/week), subcutaneous, Alzet pump 4 weeks was implanted in D1 before adenine administration

Group 6—Cilostazol (C₂₀H₂₇N₅O₂) at 45 mg/kg/day, oral, once a day

Uremia and limb ischemia were induced in groups 2-6 by a daily dose of adenine (500 mg/kg, suspended in 1% carboxymethyl cellulose, administered orally) for the first 10 days, and then with a dose of α-calcidol (100 ng/kg in olive oil, administered orally) three times per week from D11 to D19, to accelerate and homogenize the development of cardiovascular calcification and ischemia.

On day 21, animals were sacrificed, and blood and tissue samples were collected. Creatinine (Ref no. OSR6178) and urea (Ref. no. OSR6134) levels in serum were determined using the corresponding Beckman Coulter assay kit (Beckman Coulter, Inc., Brea, Calif., US).

The sham group 1 animals were administered 1% carboxymethyl cellulose solution (5 mL/kg) orally every day from D1 to D10 and then were administered orally olive oil three times per week from D11 to D19. Neither uremia nor ischemia were induced in sham group.

2. Ischemia Rescue and Effects on Blood Perfusion—Laser Doppler Imaging Assay

The subjects in groups 2a were administered physiological saline solution (2 mL/kg) subcutaneously twice a day during 21 days. The subjects in group 2b were administered physiological saline solution via subcutaneous 4 weeks using an Alzet pump.

In addition, animals in group 3 and 4 were administered subcutaneously twice a day at 30 mg/kg and 45 mg/kg of SNF472 (Na₆IP₆, free base: 600 g/mol) sodium salt (free base 696.27 g/mol), respectively. SNF472 was administrated in physiological saline solution (2 mL/kg) twice a day from D1 to D21 via subcutaneous route. The animals in group 5 were administered SNF472 at 400 mg/4 weeks dissolved physiological saline solution via subcutaneous 4 weeks Alzet pump.

Animals in group 6 were administered orally 45 mg/kg cilostazol (C₂₀H₂₇N₅O₂, free base 369.46 g/mol; lot no. LRAB9590, Sigma-Aldrich Corp., St. Louis, Mo., US) in a 5% CMC sodium salt in water solution (5 mL/kg) daily from D1 to D21.

On the days when blood perfusion tests involving the use of active agents were conducted (i.e., D10, D17, and D21), the dosages were administered as follows:

Groups 3 and 4—SNF472 (Na₆IP₆): 15 minutes before reading

Group 6—Cilostazol (C₂₀H₂₇N₅O₂): 3 to 4 hours before reading

Under the scheme above the active agents would be at their maximum plasma concentration (Cₘₐₓ) at the time when the test readings were taken.

Limb ischemia status was evaluated during treatment (i.e., D0, D10 and D17 and D21) in all rats by laser doppler perfusion imaging. The perfusion difference and perfusion ratio were calculated by comparing the baseline and of the D10, D17 and D21 readings for each group. In particular, the perfusion difference and perfusion ratio were calculated by comparing the group 1 (control) and (a) groups 2a and 2b placebo, (b) groups 3, 4 and 5 (SNF472) and (c) group 6 (cilostazol), readings.

SNF472 attenuated rat limb ischemia in uremic rats in a dose-response manner. Treatment with cilostazol alone was not effective in this model.

3. Calcium Content and Calcification—ICP-OES

Subjects were anesthetized and their blood was obtained in D21. The subjects were sacrificed after exsanguination. Then, their necropsies were performed, and their heart and aorta arteries were collected. The tissues were lyophilized for 24 h and weighed. The lyophilized tissues were then digested using a 1:1 HNO₃:HClO₄ mixture in a dry bath incubator for 2-4 h at 180° C. The digested tissues were subsequently diluted using ultra-pure Milli-Q water (MilliporeSigma (Merck KGaA), Burlington, Mass., US) to a final volume of 10 mL. The calcium content in the tissue samples was quantified via ICP-OES.

SNF472 showed to be effective against heart and vascular calcification in uremic rats, such as, for example, in heart and aorta arteries calcification, in a dose-response manner after daily subcutaneous dosing or/and delivered by Alzet pump compared to placebo. Cilostazol was not active against calcification at the same dose.

1. A method of increasing tissular perfusion and/or oxygenation in a subject in need thereof comprising administering a compound of general formula I, or a pharmaceutically acceptable salt thereof to the subject 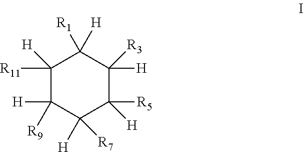
wherein R₁, R₃, R₅, R₇, R₉ and R₁₁ are independently selected from OH, a radical of formula II, III, IV and a heterologous moiety: 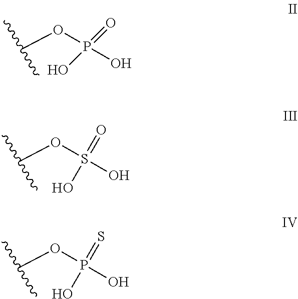
and wherein:
(i) at least one of R₁, R₃, R₅, R₇, R₉ and R₁₁ is selected from a radical of formula II, III and IV, and
(ii) zero, one, two or three of R₁, R₃, R₅, R₇, R₉ and R₁₁ is a heterologous moiety.

2. (canceled)

3. The method of claim 1, wherein the heterologous moiety is selected from a radical of formula V, a radical of formula VI and a radical of formula VII: 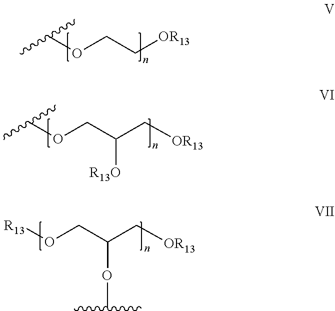
wherein: n is an integer in the range from 2 to 200, and
R₁₃ is selected from H, methyl and ethyl.

4. The method of claim 1, wherein the subject suffers from peripheral arterial disease or is at risk of developing peripheral arterial disease.

5. The method of claim 1, wherein the subject suffers from critical limb ischemia or is at risk of developing critical limb ischemia.

6. The method of claim 1, wherein the pharmaceutically acceptable salt is a sodium salt.

7. The method of claim 1, wherein the compound of formula I is inositol hexaphosphate.

8. The method of claim 7, wherein the inositol hexaphosphate is myo-inositol hexaphosphate.

9. The method of claim 7, wherein the inositol hexaphosphate is a hexasodium salt.

10. The method of claim 1, wherein one or two of R₁, R₃, R₅, R₇, R₉ and R₁₁ is selected from a radical of formula V, VI and VII.

11. The method of claim 10, wherein R₁, R₅, R₉ and R₁₁ are a radical of formula II and R₃ and R₇ are a radical of formula V.

12. The method of claim 11, wherein the radical of formula V has n in the range from 2 to 200 and R₁₃ is H.

13. The method of claim 1, wherein the compound or pharmaceutically acceptable salt thereof is in a pharmaceutical composition comprising pharmaceutically acceptable excipients and carriers.

14. The method of claim 1, wherein the subject is a subject with kidney failure.

15. The method of claim 1, wherein the compound or pharmaceutically acceptable salt thereof is administered to the subject during dialysis.

16. The method of claim 1, wherein the compound or pharmaceutically acceptable salt thereof is administered to the subject during hemodialysis.

17. The method of claim 1, wherein the compound or pharmaceutically acceptable salt thereof is administered to an unfiltered blood extracted from the subject.

18. The method of claim 1, wherein the compound or pharmaceutically acceptable salt thereof is administered by parenteral route.

19. The method of claim 18, wherein the parenteral route is intravenous, subcutaneous or intramuscular.

20. (canceled)

21. (canceled)

22. The method of claim 1, wherein the compound or pharmaceutically acceptable salt thereof is administered to the subject in an effective dosage of about 0.001 mg/kg to about 60 mg/kg.

23. A method of treating or preventing ischemia and/or an ischemia-related disease or condition in a subject in need thereof, comprising administering a compound of general formula I, or a pharmaceutically acceptable salt thereof to the subject 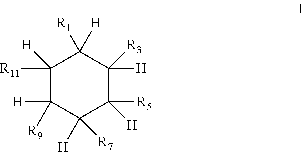
wherein R₁, R₃, R₅, R₇, R₉ and R₁₁ are independently selected from OH, a radical of formula II, III, IV and a heterologous moiety: 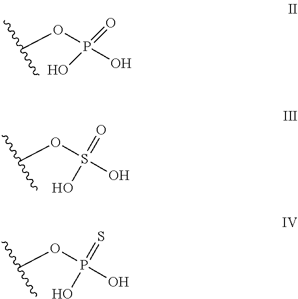
wherein:
(i) at least one of R₁, R₃, R₅, R₇, R₉ and R₁₁ is selected from a radical of formula II, III and IV, and
(ii) zero, one, two or three of R₁, R₃, R₅, R₇, R₉ and R₁₁ is a heterologous moiety,
and wherein the subject suffers from peripheral arterial disease or is at risk of developing peripheral arterial disease, the subject suffers from critical limb ischemia or is at risk of developing critical limb ischemia, the subject has kidney failure or is at risk of suffering kidney failure, or the subject is undergoing hemodialysis.

24. The method of claim 23, wherein the ischemia is limb ischemia.

25. A method of improving the walking ability of a subject in need thereof, comprising administering a compound of general formula I, or a pharmaceutically acceptable salt thereof to the subject 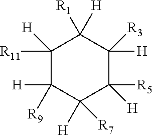
wherein R₁, R₃, R₅, R₇, R₉ and R₁₁ are independently selected from OH, a radical of formula II, III, IV and a heterologous moiety: 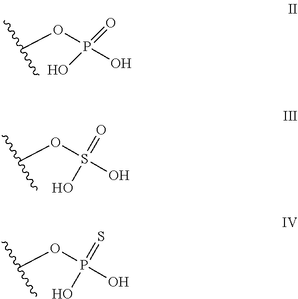
wherein:
(i) at least one of R₁, R₃, R₅, R₇, R₉ and R₁₁ is selected from a radical of formula II, III and IV, and
(ii) zero, one, two or three of R₁, R₃, R₅, R₇, R₉ and R₁₁ is a heterologous moiety,
and wherein the subject suffers from peripheral arterial disease or is at risk of developing peripheral arterial disease, the subject suffers from critical limb ischemia or is at risk of developing critical limb ischemia, the subject has kidney failure or is at risk of suffering kidney failure, or the subject is undergoing hemodialysis.

26. The method of claim 25, wherein the increase in limb function improves the walking ability of the subject.

27. The method of claim 25, wherein the increase in limb function is determined by Maximal Walking Distance (MWD), Maximal Walking Time (MWT) or both.

28. The method of claim 1, wherein the compound is SNF472.

29. The method of claim 1, wherein the compound is administered as a dose between 2.5 mg/kg and 5 mg/kg, between 5 mg/kg and 7.5 mg/kg, between 7.5 mg/kg and 10 mg/kg, or between 5 mg/kg and 10 mg/kg.

30. The method of claim 1, wherein the compound is administered at 300 mg per dose.

31. A method to treat or inhibit the development of peripheral arterial disease or critical limb ischemia in a subject comprising administering to the subject a therapeutically effective dose of inositol hexaphosphate between 5 mg/kg and 10 mg/kg.

32. The method of claim 31, wherein the dose of inositol hexaphosphate is between 7.5 mg/kg and 10 mg/kg.

33. The method of claim 31, wherein the dose of inositol hexaphosphate is administered intravenously or subcutaneously.
